(19) United States
(12) Patent Application Publication
(10) Pub. No.: US 2025/0025411 A1
(43) Pub. Date: Jan. 23, 2025

(54) READILY SOLUBLE AND THERMOSTABLE GLUCAGON FORMULATIONS AND DELIVERY FOR MINI-DOSING AND CLOSED -LOOP PROPHYLACTIC TREATMENT OF HYPOGLYCEMIA

(71) Applicants: Massachusetts Institute of Technology, Cambridge, MA (US); The Brigham and Women's Hospital, Inc., Boston, MA (US)

(72) Inventors: Jason Siu-Wei Li, Cambridge, MA (US); Robert S. Langer, Newton, MA (US); Carlo Giovanni Traverso, Newton, MA (US)

(73) Assignees: Massachusetts Institute of Technology, Cambridge, MA (US); The Brigham and Women's Hospital, Inc., Boston, MA (US)

(21) Appl. No.: 18/777,479

(22) Filed: Jul. 18, 2024

Related U.S. Application Data
(60) Provisional application No. 63/514,905, filed on Jul. 21, 2023.

(51) Int. Cl. A61K 9/00; A61K 9/70; A61K 38/44; C12N 9/04; C12N 9/08

(52) U.S. Cl. CPC A61K 9/0021; A61K 9/703; A61K 38/44; C12N 9/0006; C12N 9/0065; C12Y 101/03004; C12Y 111/01006

(57) ABSTRACT

Insulin therapy revolutionized the care of patients with diabetes, yet insulin-induced hypoglycemia remains a serious life-threatening complication of insulin therapy. Glucagon is a highly effective treatment for hypoglycemia; however, current dosage forms remain under-utilized due to poor patient compliance. High-density, readily soluble, and thermostable solid glucagon formulations applied with painless, application-specific microneedle-patches can treat hypoglycemia in type 1 diabetes patients who are awake or asleep. On-demand patches can prevent or treat mild hypoglycemia during the day, and enzyme-driven hypoglycemia-responsive patches can release glucagon autonomously during the night. These patches have excellent in vitro glucagon stability, loading, and release kinetics and can treat hypoglycemia in diabetic humans and animals. These delivery systems enable new modes of glucagon therapy, thereby expanding the clinical role of glucagon beyond the emergency setting.

### RELATED APPLICATIONS

This application claims priority under 35 U.S.C. § 119(e) to U.S. Provisional Application No. 63/514,905, filed Jul. 21, 2023, and entitled “Readily Soluble and Thermostable Glucagon Formulations and Delivery for Mini-Dosing and Closed-Loop Prophylactic Treatment of Hypoglycemia,” which is incorporated herein by reference.

### BACKGROUND

Hypoglycemia is a life-threatening condition that is prevalent in people with diabetes due to over-administration of insulin. This acute condition can cause debilitating mental or physical symptoms and may progress to loss of consciousness or death. Hypoglycemia remains the most serious acute complication among people with diabetes and often limits the use of intensive insulin therapy resulting in an increased risk of long-term diabetic complications.

The importance of timely treatment of severe and symptomatic hypoglycemia has long been recognized. It is becoming evident that rapid treatment of mild asymptomatic events is vital to prevent dysregulation of the body's counter regulatory hormone response and progressive blunting of the brain's response to low glucose levels, both of which can lead to a vicious cycle of recurrent hypoglycemia. Preventing mild hypoglycemia progression to severe hypoglycemia is also critical because people who experience severe hypoglycemia are at greater risk of all-cause mortality in the short term after hypoglycemic episodes. However, approved glucagon treatments for mild hypoglycemia (e.g., mini-dose glucagon therapy) are not available despite clinical evidence suggesting their effectiveness and user preference. Furthermore, the use of glucagon for the treatment of severe hypoglycemia remains under-utilized due to the pain and complexity associated with current glucagon interventions. Currently, FDA-approved prophylactic treatments for hypoglycemia and counter-regulatory closed-loop systems do not exist.

The insoluble nature of glucagon and its tendency to rapidly degrade and form insoluble fibrils in solutions has prevented the development of new glucagon formulations for many years. The recent introduction of solution-stable glucagon formulations improves upon earlier emergency glucagon rescue kits since solution-stable glucagon formulations are thermostable during storage and ready for administration through injection. However, pain and fear associated with needle injections remain a significant clinical challenge resulting in suboptimal use of an otherwise effective medication, particularly in non-emergent situations. Intranasal glucagon formulations circumvent this limitation; however, drawbacks to intranasal glucagon formulations include painful respiratory side-effects, delayed resolution to hypoglycemia, and inaccurate dose administration. These drawbacks precludes the use of intranasal glucagon formulations outside of emergencies and for treatment of mild hypoglycemia. Furthermore, the introduction of these ready-to-use injectable or inhalable glucagon formulations has not been successful at expanding the use of glucagon in patients, and their low drug density limits efficient loading and delivery via more patient-friendly ingestible or transdermal delivery devices. Thus, the insoluble and unstable nature of glucagon continues to stifle the development and availability of painless and well-tolerated glucagon treatments for a variety of hypoglycemic conditions beyond severe hypoglycemia.

### SUMMARY

Here, we disclose high-density readily soluble and thermostable (ReST) solid glucagon formulations that enable the storage and administration of glucagon in the dry form. We also disclose glucagon delivery systems that use these ReST solid glucagon formulations or other glucagon formulations or analogues, including: (1) a space-efficient, painless, and convenient microneedle patch for accurate mini-dose glucagon treatment of mild hypoglycemia and scalable for larger doses; (2) an enzymatic glucose-responsive wearable patch that provides automated and glucose-specific prophylactic treatment, for example, of nocturnal hypoglycemia throughout the night; and (3) enzymatic glucose-responsive implants that provide automated and glucose-specific prophylactic treatment of hypoglycemia. These delivery systems demonstrate the potential of the high-density ReST glucagon formulations to enable new modes of glucagon therapy, thereby expanding the clinical role of glucagon beyond the emergency setting, facilitating more widespread management of hypoglycemia, and improving the glycemic management of people with diabetes.

Embodiments of the current technology include a glucose-responsive system for delivering glucagon to a mammalian subject. Examples of the glucose-responsive system include: a polymeric release structure including: a readily soluble and thermostable (ReST) glucagon formulation; at least one of glucose oxidase, a glucose oxidase derivative, or a glucose oxidase analogue; and a pH-responsive polymer. The polymeric release structure swells and/or dissolves at a pH level of about 6 or greater and the glucagon is released from the polymeric release structure when the mammalian subject has a glucose concentration of about 100 milligrams per deciliter or less (which corresponds to a pH level of about 6 or greater).

Generally, derivatives include fragments or mutants of the native peptide/protein, and analogues include molecules with a similar function. Analogues of glucagon induce an increase in glucose in the bloodstream, including via gluconeogenesis or release from storage. Analogues of glucose oxidase metabolize glucose resulting in a change local pH.

The ReST glucagon formulation consists essentially of an excipient and at least one of glucagon, a glucagon analog, or a glucagon derivative. In addition to the excipient and glucagon, glucagon derivative, or glucagon analogue, each of the ReST glucagon formulations disclosed herein may also include stabilizers, fillers, imaging/contrast agents, etc. The ReST glucagon formulation can include about 0.2 mg to about 2.0 mg of the at least one of the glucagon, the glucagon analog, or the glucagon derivative and/or about 10% to about 90% of the at least one of the glucagon, the glucagon analog, or the glucagon derivative. The excipient can include benzoic acid, sodium carbonate, meglumine, sodium phosphate tribasic, myristyl sulfobetaine (MSB), Kollidon HS 15, dodecylphosphocholine, L-Glutamine, PIPES, or sodium succinate.

The glucose oxidase, glucose oxidase derivative, or glucose oxidase analogue may be incorporated into the pH-responsive polymer.

The polymeric release structure can include at least one of catalase, a catalase derivative, a catalase analogue, or MnO₂. Analogues of catalase have peroxidase activity and use a peroxide, e.g., hydrogen peroxide, as a substrate, resulting in the generation of oxygen.

The pH-responsive polymer can include shellac, methyl acrylate-methacrylic acid copolymers, cellulose acetate phthalate, cellulose acetate succinate, hydroxypropyl methyl cellulose phthalate, hydroxypropyl methyl cellulose acetate succinate, polyvinyl acetate phthalate, methyl methacrylate-methacrylic acid copolymers, cellulose acetate trimellitate, or zein.

The glucose-responsive system may further include a hydrogel layer loaded with the glucose oxidase and disposed on the pH-responsive polymer.

The pH-responsive polymer can form or include an enteric barrier film configured to swell and/or dissolve at the pH level of about 6 or greater. The enteric barrier film can have a thickness of about 10 microns to about 400 microns.

The glucose oxidase can catalyze production of hydrogen peroxide and gluconic acid in the presence of glucose and oxygen, thereby keeping the pH level below 6 and preventing the enteric barrier film from swelling or dissolving. In these examples, the polymeric release structure can include catalase, catalase derivative, catalase analogue, or MnO₂ to catalyze production of oxygen from the hydrogen peroxide.

The polymeric release structure can be formed into a microneedle of a transdermal microneedle patch. The glucagon is released from at least one of an interior bore of the microneedle or an outer surface of the microneedle. Alternatively, the polymeric release structure can be formed into an implantable structure or microparticle configured to be implanted into the mammalian subject beneath a stratum cornea of the mammalian subject using a hypodermic needle or trocar.

In some aspects, the techniques described herein relate to a method of delivering glucagon to a mammalian subject with a release structure including a readily soluble and thermostable (ReST) glucagon formulation contained within a pH-sensitive polymer that incorporates or is coated with glucose oxidase and that swells and/or dissolves at a pH of about 6 or greater, the method including: inserting the release structure in the mammalian subject or applying the release structure to skin of the mammalian subject. The glucose oxidase catalyzes production of gluconic acid and hydrogen peroxide in the presence of glucose and oxygen, thereby keeping the pH of the pH-sensitive polymer below 6 in the presence of glucose (e.g., a glucose concentration of more than 100 milligrams per deciliter) and preventing release of the ReST glucagon formulation from the release structure. The pH of the pH-sensitive polymer rises above 6 in the absence of glucose (e.g., a glucose concentration of 100 milligrams per deciliter or less), causing the pH-sensitive polymer to swell and/or dissolve, thereby releasing the ReST glucagon formulation from the release structure (e.g., at a rate of about 1 mg in 30 seconds to about 1 mg in 4 hours). The release structure may further include catalase, catalase derivative, catalase analogue, or MnO₂ to catalyze production of oxygen from the hydrogen peroxide.

In some aspects, the techniques described herein relate to a readily soluble and thermostable glucagon formulation consisting essentially of: at least one of glucagon, a glucagon analog, or a glucagon derivative; and an excipient from the group consisting of benzoic acid, sodium carbonate, meglumine, sodium phosphate tribasic, myristyl sulfobetaine, Kollidon HS 15, dodecylphosphocholine, L-Glutamine, PIPES, or sodium succinate.

All combinations of the foregoing concepts and additional concepts discussed in greater detail below (provided such concepts are not mutually inconsistent) are part of the inventive subject matter disclosed herein. In particular, all combinations of claimed subject matter appearing at the end of this disclosure are part of the inventive subject matter disclosed herein. The terminology used herein that also may appear in any disclosure incorporated by reference should be accorded a meaning most consistent with the particular concepts disclosed herein.

### BRIEF DESCRIPTIONS OF THE DRAWINGS

The skilled artisan will understand that the drawings primarily are for illustrative purposes and are not intended to limit the scope of the inventive subject matter described herein. The drawings are not necessarily to scale; in some instances, various aspects of the inventive subject matter disclosed herein may be shown exaggerated or enlarged in the drawings to facilitate an understanding of different features. In the drawings, like reference characters generally refer to like features (e.g., functionally and/or structurally similar elements).

FIG. 1A shows results of an in vitro screen to identify solid excipients capable of enhancing glucagon dissolution, with the concentration of solubilized glucagon following a 30-minute incubation of lyophilized glucagon in pH 7.4 phosphate-buffered saline (PBS) solutions containing 1 mg/mL of excipient for each excipient (n≥3; * denotes statistically significant differences compared to a PBS control).

FIG. 1B is a photograph of 1 mm diameter tablets formed of high-density, compressed solid glucagon formulations composed of myristyl sulfobetaine (MSB) and glucagon, one of which is loaded within a 16 G hypodermic needle.

FIG. 1C is a plot of glucagon release kinetics of solid glucagon tablets containing lyophilized glucagon and MSB at various ratios (n≥3).

FIG. 1D is a plot of stability (i.e., chemical purity) of acute glucagon formulations under various conditions (i.e., freshly prepared, dried, and following storage) (n≥3).

FIG. 1E is a plot of stability (i.e., chemical purity) of responsive glucagon formulations under various conditions (i.e., freshly prepared, dried, and following storage) (n≥3).

FIG. 1F illustrates a scheme for evaluating the bioactivity of glucagon formulations in diabetic rats.

FIG. 1G is a plot of hyperglycemic activity of glucagon formulations administered to diabetic rats (n=3).

FIG. 2A illustrates a transdermal microneedle patch for glucagon mini dosing.

FIG. 2B shows photographs of the mini-dosing microneedle patch of FIG. 2A before immersion in PBS (top) and after immersion in PBS for 30 seconds (upper middle), 60 seconds (lower middle), and 120 seconds (bottom).

FIG. 2C is a plot of glucagon loading within the microneedle tips of a microneedle patch for glucagon mini dosing (n≥4).

FIG. 2D illustrates a scheme for testing pharmacokinetics and pharmacodynamics of microneedle patches for glucagon mini dosing in diabetic rats.

FIG. 2E is a photograph of rat skin following application of a mini-dosing microneedle patch.

FIG. 2F shows a hematoxylin and eosin (H&E) stained tissue section showing microneedle penetration through the stratum corneum of pig skin.

FIG. 2G shows the change in glucose levels following transdermal application of mini-dosing microneedle patches loaded with different levels of glucagon, compared to saline control and subcutaneous (SC) injection of a glucagon solution.

FIG. 2H is a plot of area-under-the-curve (AUC) of blood glucose levels following transdermal application of mini-dosing microneedle patches loaded with different levels of glucagon, compared to saline control and SC injection of glucagon solution.

FIG. 2I is a plot of change in plasma glucagon levels following transdermal application of mini-dosing microneedle patches loaded with different levels of glucagon, compared to saline control and SC injection of glucagon solution.

FIG. 2J is a plot of AUC of plasma glucagon levels following transdermal application of mini-dosing microneedle patches loaded with different levels of glucagon, compared to saline control and SC injection of glucagon solution.

FIG. 2K shows AUC, Cₘₐₓ, Tₘₐₓ, and rate of blood glucose level change following treatment with a mini-dosing microneedle patch loaded with 300 μg of glucagon or an equivalent dose administered via subcutaneous injection.

FIG. 3A shows a transdermal, glucose-responsive microneedle patch for prophylactic hypoglycemia treatment using hypoglycemia-triggered glucagon delivery.

FIG. 3B shows confocal microscopy images of the microneedle structure of the transdermal microneedle patch of FIG. 3A. The enzyme-functionalized hydrogel microneedle structure is highlighted in the left two images. The enteric film is highlighted in the middle right image. And the ReST glucagon formulation is highlighted in the right-most image.

FIG. 3C is a photograph of pig skin following application of the transdermal glucose-responsive microneedle patch of FIG. 3A. Punctuated dots show the penetration of methylene blue dye into the skin following microneedle application.

FIG. 3D shows an H&E-stained tissue section showing microneedle penetration through the stratum corneum of pig skin.

FIG. 3E shows in vitro glucagon release kinetics from glucose-responsive microneedle patches with 20, 40, and 60 μm thick enteric polymer films in buffers with pHs of 3.0 (lower traces) and 7.4 (upper traces) (n≥3).

FIG. 3F shows in vitro glucagon release times from glucose-responsive microneedle patches with 20, 40, and 60 μm thick enteric polymer films in buffers with pHs of 3.0 (right bars) and 7.4 (left bars) (n≥3).

FIG. 3G shows in vitro glucagon release kinetics from glucose-responsive microneedle patches with 20, 40, and 60 μm thick enteric polymer films in pH 7.4 PBS supplemented with glucose at concentrations of 50 mg/dL (upper traces) and 200 mg/dL (lower traces) (n≥3).

FIG. 3H shows in vitro glucagon release times from glucose-responsive microneedle patches with 20, 40, and 60 μm thick enteric polymer films in pH 7.4 PBS supplemented with glucose at concentrations of 50 mg/dL (left bars) and 200 mg/dL (right bars) (n≥3).

FIG. 3I illustrates a scheme for pharmacokinetic and pharmacodynamic evaluation of prophylactic hypoglycemia treatment in diabetic rats using glucose-responsive microneedle patches.

FIG. 3J shows photographs of a glucose-responsive microneedle patch for prophylactic hypoglycemia treatment in a diabetic rat during and after transdermal application.

FIG. 3K is a plot of blood glucose levels before transdermal application of the glucose-responsive glucagon patch. Insulin was administered at 0 hours. The glucose-responsive microneedle patch was applied at 3 hours.

FIG. 3L is a plot of blood glucose levels after transdermal application of the glucose-responsive glucagon patch. Insulin was administered at 0 hours. The glucose-responsive microneedle patch was applied at 3 hours.

FIG. 3M is a plot of plasma glucagon levels before transdermal application of the glucose-responsive glucagon patch. Insulin was administered at 0 hours. The glucose-responsive microneedle patch was applied at 3 hours.

FIG. 3N is a plot of plasma glucagon levels after transdermal application of the glucose-responsive glucagon patch. Insulin was administered at 0 hours. The glucose-responsive microneedle patch was applied at 3 hours.

FIG. 4A shows enteric-coated, solid glucagon microparticles with hydrogel coatings for prophylactic treatment of hypoglycemia.

FIG. 4B shows an enteric-coated, solid glucagon implant with a hydrogel coating for prophylactic treatment of hypoglycemia.

FIG. 4C shows a solid glucagon implant with a pH-sensitive enteric polymer coating and enzymes on or incorporated into the enteric polymer coating for prophylactic treatment of hypoglycemia.

FIG. 4D shows a tethered, enteric-coated solid glucagon implant with a hydrogel coating for prophylactic treatment of hypoglycemia.

FIG. 5A shows a table of various characteristics associated with participants having type 1 diabetes, according to some embodiments.

FIG. 5B shows a table describing the incidence of hypoglycemic events, according to some embodiments.

FIG. 5C shows a table describing characteristics of parent hypoglycemic events, according to some embodiments.

FIG. 6A shows the extraction and parameterization of hypoglycemic events from raw CGM trace, according to some embodiments.

FIG. 6B depicts Sankey diagrams showing the classification of hypoglycemic events, according to some embodiments.

FIG. 6C depicts proportions of complete initial and follow-on events in the general and well-controlled T1D populations, according to some embodiments.

FIG. 6D depicts the incidence of parent events by several associated follow-on events, according to some embodiments.

FIG. 6E depicts the proportion of complete parent events by hypoglycemic severity and time of occurrence, according to some embodiments.

FIG. 6F depicts the probability density of hypoglycemia durations by hypoglycemia severity and time of day, according to some embodiments, where average duration (center vertical line) and 25th and 75th percentiles (outer vertical lines) are indicated in each plot.

FIG. 6G depicts the probability density of time-to-treatment following the onset of hypoglycemia, according to some embodiments, where average time (center vertical line) and 25th and 75th percentiles (outer vertical lines) are indicated in each plot.

FIG. 7 depicts a process workflow of CGM data with a) data workflow and b) state machine diagram, according to some embodiments.

FIG. 8 depicts the incidence of complete parent events by time of occurrence, according to some embodiments.

FIG. 9 depicts CGM time traces a) Average (dots, 25th and 75th percentile (shaded areas) of daytime (light) and nighttime (dark) CGM traces during pre-hypoglycemia, according to some embodiments. b) Average (dots), 25th and 75th percentile (shaded areas) of daytime (light) and nighttime (dark) CGM traces during hypoglycemia.

FIG. 10 depicts a violin plot of the hypoglycemic event duration in patients with general and well-controlled T1D, according to some embodiments.

FIG. 11 depicts a violin plot of the time to treatment for in patients with general and well-controlled T1D, according to some embodiments.

FIG. 12 depicts boxplots of the rate onset into hypoglycemia (generated data) with the original rate onset into hypo durations (raw data; circular dots) plotted for reference, according to some embodiments.

FIG. 13 depicts boxplots of the pre-hypoglycemia durations (generated data) with the original pre-hypoglycemia durations (raw data; circular dots) plotted for reference, according to some embodiments.

FIG. 14 depicts boxplots of the durations of mild hypo preceding severe hypo (generate data) with the original durations (raw data; circular dots) plotted for reference, according to some embodiments.

FIG. 15 depicts boxplots of the pre-hypoglycemia durations (generated data) with the original pre-hypoglycemia durations (raw data; circular dots) plotted for reference, according to some embodiments.

FIG. 16 depicts boxplots of the durations of hypoglycemia until the first glucagon treatment (generated data) with the original data (raw data; circular dots) plotted for reference, according to some embodiments.

FIG. 17 depicts boxplots of the durations of hypoglycemia until the last glucagon treatment (generated data) with the original durations (raw data; circular dots) plotted for reference, according to some embodiments.

FIG. 18 depicts glucagon solubility versus pH of excipient solutions, according to some embodiments. pH values were measured using a digital pH meter equipped with a micro pH electrode. Solutions were prepared at a concentration of 1 mg/mL in water.

FIG. 19 depicts A) a plot describing the solubility of dried glucagon formulations, according to some embodiments, and B) a photograph of dried glucagon formulations 10 minutes after reconstitution in 2 mL of pH 7.4 PBS.

FIG. 20 depicts a plot describing the solubility of dried glucagon formulations, according to some embodiments.

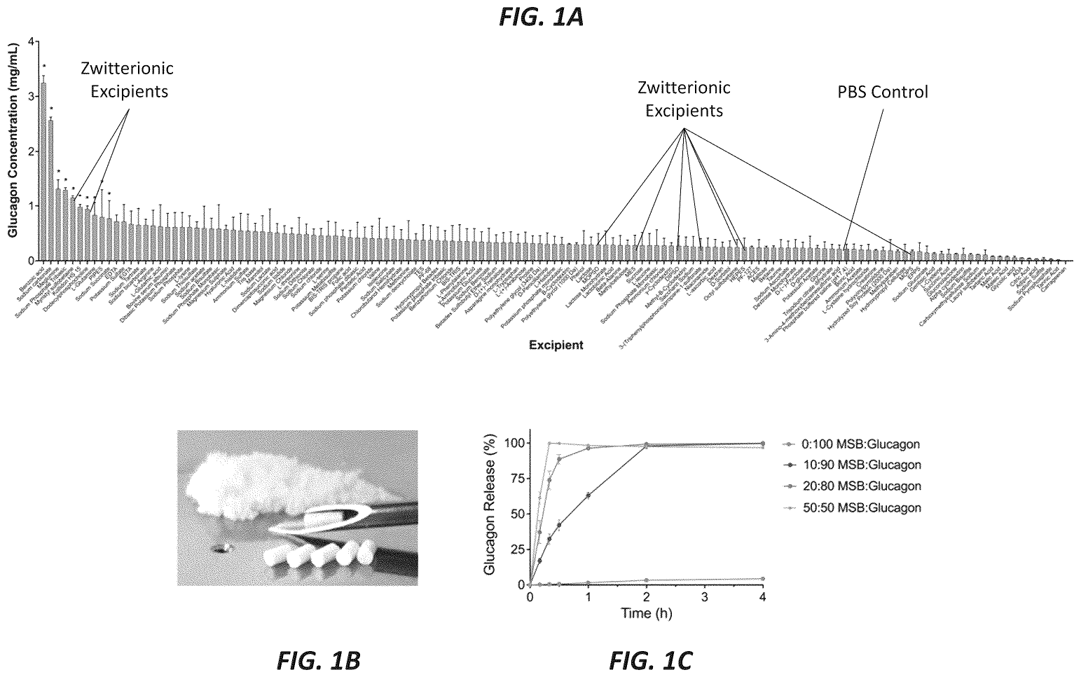

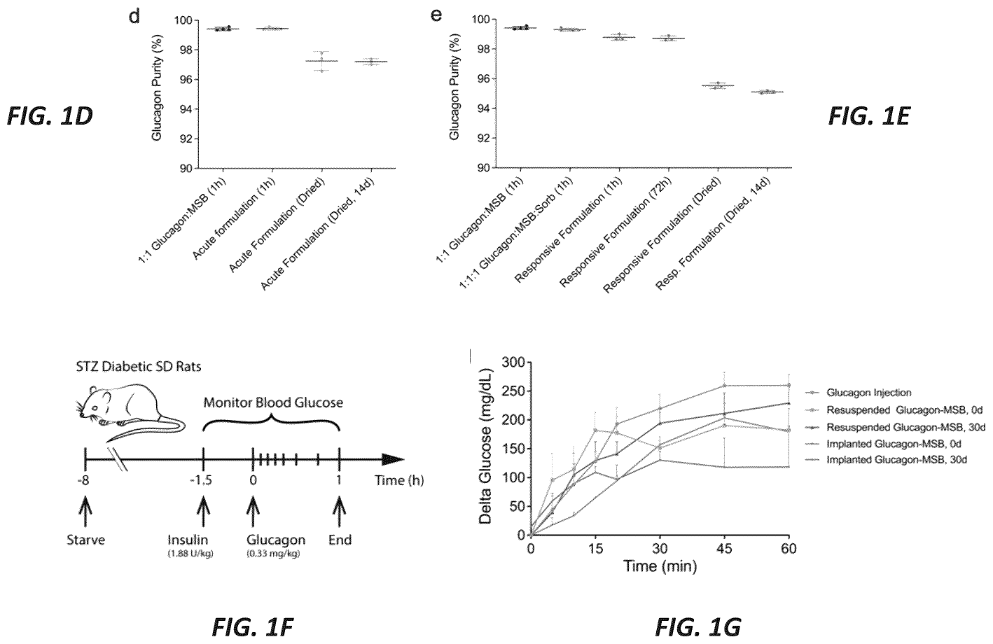

Data are presented as mean±standard deviation (SD) in FIGS. 1A-1E, 2C, and 3E-3H and as mean±standard error of the mean (SEM) FIGS. 1G, 2G-2J, and 3K-3N. Experimental replicates are indicated where applicable. Statistically significant differences indicated as * for p<0.05.

### DETAILED DESCRIPTION

High-density, solid glucagon formulations that are readily soluble and thermo-stable (ReST) can be used for treating hypoglycemia in people with type 1 diabetes outside the emergency setting, for example, in a home setting. These treatments include a transdermal, mini-dosing microneedle patch for treating mild hypoglycemia painlessly, conveniently, and accurately. The mini-dosing microneedle patch includes a solid ReST glucagon formulation mixed with a polymer matrix and dried to form an array of xerogel (dry hydrogel) microneedles on a flexible adhesive backing. Applying the mini-dosing microneedle patch to a person's skin causes the microneedle to absorb moisture, transforming the xerogel into hydrogel and releasing the glucagon into the person's skin, where it is taken up in the person's bloodstream.

Other treatments with ReST glucagon formulations include a glucose-responsive microneedle patch for prophylactic treatment of hypoglycemia suitable for use in just about any setting, including a home setting. This glucose-responsive microneedle patch includes microneedles loaded with a ReST glucagon formulation and coated with an inner pH-sensitive enteric polymer coating and an outer hydrogel coating containing glucose oxidase and catalase enzymes. The glucose-responsive microneedle patch is applied to person's skin, e.g., before the person goes to sleep. Its glucose-responsive glucagon release mechanism provides hypoglycemia-responsive drug release via a glucose-specific transduction moiety and prevents undesired glucagon elution under euglycemic conditions. In other words, the glucose-responsive microneedle patch releases glucagon when the person's glucose levels are low, but not when they are normal or high.

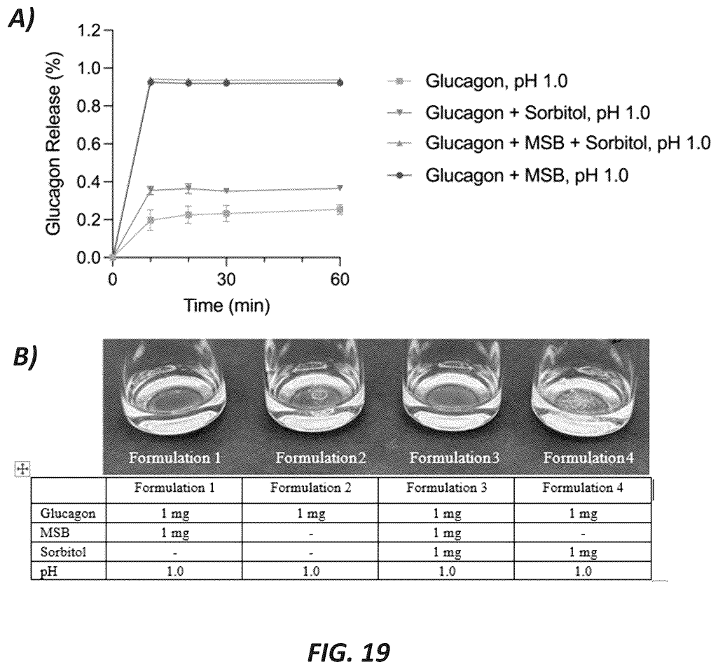

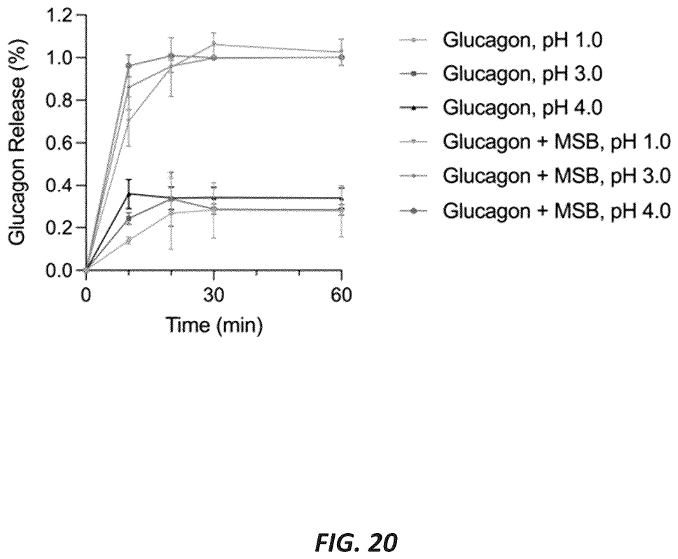

More generally, the rate at which the glucose-responsive microneedle patch releases glucagon varies with the person's glucose level, with high release rates at lower glucose levels and lower release rates at higher glucose levels. Release rates and time-to-release may be faster for higher pH and conversely slower for lower pH. The release rates can be tuned by varying the thickness and chemistry of a pH-sensitive enteric polymer coating that encapsulates the glucagon formulation. Typically, a glucose-responsive microneedle patch may release glucagon within an hour (the faster the better) at a higher pH (e.g., a pH above 6.0); and prevent glucagon release for at least 4 to 8 hours (the longer the better) at lower pH (e.g., a pH below 5.0). Additional glucagon release data can be found in FIGS. 19-20. In FIG. 19, formulations consist of glucagon formulated with or without MSB and sorbitol and solubilized in pH 1.0 water. Glucagon was soluble in all formulations prior to air drying at room temperature for 24 hours. Glucagon dissolution rates were measured following reconstitution in 2 mL of pH 7.4 PBS using RP-HPLC. Referring to FIG. 20, glucagon within formulations containing MSB were fully dissolved. Insoluble glucagon precipitates were observed in formulations without MSB.

When the person's glucose levels are normal or high, the available glucose drives enzymatic reactions catalyzed by glucose oxidase and catalase in the hydrogel coating. These reactions produce gluconic acid, which maintains an acidic microenvironment within the hydrogel matrix, keeping the enteric polymer coating intact and preventing release of the ReST glucagon formulation. When the person's glucose levels become low (e.g., about 100 milligrams per deciliter or less), the reactions catalyzed by the glucose oxidase and catalase produce less gluconic acid, causing the pH within the hydrogel to rise to 6.0 or higher (e.g., to about 7.0 or about 7.4). The enteric polymer film swells or dissolves rapidly in the neutral pH, releasing the glucagon from the glucose-responsive microneedle patch.

### High-Density Solid Readily Soluble and Thermostable (ReST) Glucagon Formulations

FIG. 1A and TABLE 1 (below) show the results of a high-throughput formulation screen performed to identify solid pharmaceutical excipients capable of promoting dissolution of lyophilized glucagon in neutral pH solutions. Among the 141 excipients tested, 10 compounds successfully enhanced the dissolution of glucagon following a 30-minute incubation in pH 7.4 PBS at 37° C. (other incubation periods could be used instead) as indicated by statistical significance compared to no excipient (i.e., just pH 7.4 PBS). These excipients included: benzoic acid, sodium carbonate, meglumine, sodium phosphate tribasic, myristyl sulfobetaine (MSB), Kollidon HS 15, dodecylphosphocholine, L-Glutamine, piperazine-N,N′-bis(2-ethanesulfonic acid) (PIPES), and sodium succinate. Some of these excipients enhanced solution glucagon concentrations by up to 16-fold compared to unformulated glucagon. The successful excipients can be used to make readily soluble, high-density binary solid formulations containing as much as 80% wt. glucagon, e.g., like the 1 mm diameter tablets of a solid glucagon formulation composed of glucagon and the MSB shown in FIG. 1B.

Many of the top hits in the excipient screening (e.g., benzoic acid and sodium carbonate) promote glucagon dissolution by altering solution pH. Unlike many other successful excipients, the zwitterionic compound MSB can enhance glucagon dissolution in PBS without altering solution pH. Evaluation of the in vitro dissolution kinetics of a compacted solid glucagon-MSB formulation demonstrates the ability of MSB to promote rapid and complete dissolution of lyophilized glucagon in a dose dependent manner in neutral pH buffer compared to unformulated glucagon, which remained largely insoluble as shown in FIG. 1C.

FIGS. 1D and 1E illustrate the effect of MSB content on the stability of glucagon following storage under accelerated storage conditions (40° C. and 75% relative humidity). High-performance liquid chromatography (HPLC) analysis of different MSB-glucagon formulations following a 14-day storage period showed no significant loss in glucagon purity, as seen in FIGS. 1D and 1E. To confirm that a readily soluble thermostable (ReST) solid glucagon formulation composed of 20% MSB and 80% glucagon remained biologically active following long-term storage, we resuspended it, both prepared fresh and after a 30-day storage period at 40° C., in sterile saline and administered both preparations subcutaneously to diabetic rats at a dose of 0.1 mg glucagon as shown in FIG. 1F. FIG. 1G shows that both preparations produced a comparable therapeutic effect, both in the magnitude of blood glucose elevation and the rate of onset, suggesting no significant loss in biological activity following long-term (30-day) storage. The resulting pharmacological effects were also similar to freshly reconstituted (unformulated) lyophilized glucagon, indicating no loss in glucagon activity when formulated with MSB.

We also administered the ReST solid glucagon formulations to the subcutaneous tissue of diabetic rats as a solid injectable tablet through a 16-gauge hypodermic needle (i.e., without prior resuspension in a carrier fluid) to evaluate the kinetics of glucagon dissolution and ensuing hyperglycemia. FIG. 1G shows that administration caused an immediate increase in blood glucose levels, demonstrating the ability of these solid formulations to elicit a rapid therapeutic effect comparable to that of freshly prepared glucagon solutions.

The stability of ReST formulations was evaluated after each stage of loading within acute (FIG. 1D) and responsive (FIG. 1E) microneedle patches. Glucagon remained stable after mixing with structural microneedle materials (e.g., hyaluronic acid, gelatin, sorbitol), but exhibited a small 2-3% loss in purity following drying. Surprisingly, HPLC analysis showed no significant loss in glucagon purity after storage of the dry glucagon-loaded microneedle patches in accelerated conditions (14 days at 40° C. and 75% relative humidity) (FIG. 1D, 1E).

TABLE 1
High throughput screening of a library
of water-soluble solid excipients

| | | Glucagon | | Fold change | |
|---|---|---|---|---|---|
| | | concentration | | vs. PBS | |
| | | Avg. | | Avg. | |
| | Excipient | (mg/mL) | SD | (fold) | SD |
| 1 | Benzoic acid | 3.25 | 0.13 | 15.09 | 0.60 |
| 2 | Sodium carbonate | 2.56 | 0.06 | 11.91 | 0.29 |
| 3 | Meglumine | 1.32 | 0.17 | 6.13 | 0.77 |
| 4 | Sodium Phosphate Tribasic | 1.30 | 0.04 | 6.04 | 0.20 |
| 5 | Myristyl sulfobetaine | 1.15 | 0.04 | 5.33 | 0.21 |
| 6 | Kollidon HS 15 | 0.98 | 0.05 | 4.55 | 0.22 |
| 7 | Dodecylphosphocholine | 0.94 | 0.06 | 4.37 | 0.28 |
| 8 | L-Glutamine | 0.83 | 0.23 | 3.87 | 1.08 |
| 9 | Piperazine-N,N′-bis(2- | 0.80 | 0.51 | 3.70 | 2.37 |
| | ethanesulfonic acid) (PIPES) | | | | |
| 10 | Sodium Succinate | 0.77 | 0.32 | 3.58 | 1.50 |
| 11 | EDTA | 0.72 | 0.12 | 3.34 | 0.55 |
| 12 | Potassium Sulfate | 0.71 | 0.31 | 3.32 | 1.44 |
| 13 | EGTA | 0.67 | 0.24 | 3.11 | 1.11 |
| 14 | Sodium Tartrate | 0.65 | 0.30 | 3.04 | 1.39 |
| 15 | Sodium bicarbonate | 0.65 | 0.11 | 3.03 | 0.49 |
| 16 | L-Arginine | 0.64 | 0.28 | 2.98 | 1.30 |
| 17 | L-Glutamic acid | 0.63 | 0.39 | 2.91 | 1.82 |
| 18 | Bovine serum albumin | 0.62 | 0.25 | 2.86 | 1.16 |
| 19 | Dibasic Potassium Phosphate | 0.61 | 0.27 | 2.85 | 1.24 |
| 20 | Sodium Phosphite | 0.61 | 0.27 | 2.85 | 1.24 |
| 21 | L-lysine | 0.61 | 0.21 | 2.84 | 0.97 |
| 22 | Sodium Thiosulfate | 0.60 | 0.11 | 2.80 | 0.51 |
| 23 | Sodium acetate | 0.59 | 0.40 | 2.76 | 1.86 |
| 24 | Sodium Bisulfate | 0.59 | 0.19 | 2.72 | 0.88 |
| 25 | Sodium Phosphate Monobasic | 0.58 | 0.44 | 2.72 | 2.03 |
| 26 | Magnesium Sulphate | 0.57 | 0.08 | 2.66 | 0.36 |
| 27 | Hyaluronic Acid | 0.57 | 0.23 | 2.63 | 1.07 |
| 28 | L-histidine | 0.55 | 0.31 | 2.57 | 1.45 |
| 29 | Ammonium Sulfate | 0.54 | 0.31 | 2.52 | 1.44 |
| 30 | Tris base | 0.54 | 0.14 | 2.50 | 0.67 |
| 31 | Mannitol | 0.54 | 0.29 | 2.49 | 1.33 |
| 32 | Sodium Lactate | 0.52 | 0.42 | 2.44 | 1.95 |
| 33 | Dimercaptosuccinic acid | 0.51 | 0.17 | 2.38 | 0.77 |
| 34 | Sodium Iodide | 0.50 | 0.14 | 2.33 | 0.65 |
| 35 | Magnesium Chloride | 0.50 | 0.10 | 2.30 | 0.48 |
| 36 | L-threonine | 0.49 | 0.30 | 2.26 | 1.39 |
| 37 | Sodium Dithionite | 0.49 | 0.19 | 2.25 | 0.86 |
| 38 | Sodium Chlorate | 0.47 | 0.11 | 2.19 | 0.51 |
| 39 | Sodium chloride | 0.46 | 0.13 | 2.14 | 0.59 |
| 40 | L-serine | 0.46 | 0.26 | 2.13 | 1.21 |
| 41 | Potassium Metabisulfite | 0.46 | 0.24 | 2.13 | 1.13 |
| 42 | BIS-TRIS propane | 0.45 | 0.11 | 2.08 | 0.53 |
| 43 | Folic acid | 0.43 | 0.13 | 1.98 | 0.60 |
| 44 | Sodium phosphate dibasic | 0.42 | 0.28 | 1.95 | 1.31 |
| 45 | Pentetic Acid | 0.42 | 0.19 | 1.95 | 0.90 |
| 46 | Potassium chloride | 0.41 | 0.22 | 1.89 | 1.04 |
| 47 | Valine | 0.41 | 0.37 | 1.89 | 1.71 |
| 48 | Isoleucine | 0.41 | 0.21 | 1.88 | 0.97 |
| 49 | Sodium Salicylate | 0.40 | 0.24 | 1.84 | 1.10 |
| 50 | Chlorobutanol Hemihydrate | 0.39 | 0.17 | 1.83 | 0.81 |
| 51 | Methionine | 0.39 | 0.34 | 1.81 | 1.58 |
| 52 | Sodium deoxycholate | 0.38 | 0.12 | 1.75 | 0.54 |
| 53 | TES | 0.38 | 0.28 | 1.75 | 1.30 |
| 54 | PF-68 | 0.38 | 0.27 | 1.75 | 1.23 |
| 55 | Hydroxypropyl Betadex | 0.37 | 0.24 | 1.74 | 1.12 |
| 56 | Potassium phosphate tribasic | 0.37 | 0.18 | 1.71 | 0.85 |
| 57 | Benzethonium Chloride | 0.36 | 0.28 | 1.68 | 1.31 |
| 58 | BIS-TRIS | 0.36 | 0.30 | 1.67 | 1.39 |
| 59 | L-Phenylalanine | 0.36 | 0.23 | 1.66 | 1.06 |
| 60 | y-Aminobutyric Acid | 0.35 | 0.24 | 1.63 | 1.11 |
| 61 | Potassium Gluconate | 0.35 | 0.19 | 1.61 | 0.86 |
| 62 | Sodium Benzoate | 0.34 | 0.25 | 1.59 | 1.17 |
| 63 | Betadex Sulfobutyl Ether | 0.34 | 0.14 | 1.56 | 0.65 |
| | Sodium | | | | |
| 64 | D-(+)-Trehalose | 0.33 | 0.18 | 1.55 | 0.85 |
| 65 | Asparagine monohydrate | 0.33 | 0.20 | 1.55 | 0.92 |
| 66 | L-Tryptophan | 0.33 | 0.16 | 1.54 | 0.72 |
| 67 | L-(+)-Arabinose | 0.33 | 0.20 | 1.52 | 0.91 |
| 68 | Proline | 0.32 | 0.14 | 1.48 | 0.64 |
| 69 | Polyethylene glycol (3400 Da) | 0.32 | 0.14 | 1.47 | 0.66 |
| 70 | D-Penicillamine | 0.31 | 0.20 | 1.42 | 0.95 |
| 71 | L-Histidine | 0.30 | 0.14 | 1.41 | 0.67 |
| 72 | Potassium phosphate monobasic | 0.30 | 0.12 | 1.41 | 0.57 |
| 73 | B-Cyclodextrin | 0.30 | 0.01 | 1.40 | 0.06 |
| 74 | Polyethylene glycol (1000 Da) | 0.30 | 0.03 | 1.39 | 0.14 |
| 75 | Phenol | 0.30 | 0.25 | 1.38 | 1.17 |
| 76 | Lactose | 0.30 | 0.14 | 1.38 | 0.64 |
| 77 | MOPSO | 0.29 | 0.21 | 1.37 | 0.95 |
| 78 | Lactose Monohydrate | 0.29 | 0.25 | 1.37 | 1.16 |
| 79 | Lactobionic Acid | 0.29 | 0.13 | 1.35 | 0.61 |
| 80 | Beta-Alanine | 0.29 | 0.17 | 1.34 | 0.81 |
| 81 | Methylcellulose | 0.29 | 0.09 | 1.33 | 0.43 |
| 82 | MES | 0.29 | 0.18 | 1.32 | 0.85 |
| 83 | Sucrose | 0.28 | 0.23 | 1.32 | 1.08 |
| 84 | L-leucine | 0.28 | 0.22 | 1.32 | 1.00 |
| 85 | Sodium Phosphate Monobasic | 0.28 | 0.28 | 1.31 | 1.32 |
| 86 | Ammonium chloride | 0.28 | 0.13 | 1.31 | 0.61 |
| 87 | y-Cyclodextrin | 0.28 | 0.05 | 1.30 | 0.23 |
| 88 | DIPSO | 0.28 | 0.18 | 1.28 | 0.84 |
| 89 | Methyl-B-Cyclodextrin | 0.27 | 0.16 | 1.27 | 0.75 |
| 90 | Saccharin Sodium | 0.26 | 0.19 | 1.21 | 0.86 |
| 91 | 3-(Triphenylphosphonio) | 0.26 | 0.15 | 1.21 | 0.68 |
| | propane-1-sulfonate | | | | |
| 92 | Niacinamide | 0.25 | 0.16 | 1.17 | 0.75 |
| 93 | L-ascorbic acid | 0.25 | 0.14 | 1.17 | 0.65 |
| 94 | Dextran | 0.25 | 0.09 | 1.16 | 0.42 |
| 95 | D-Sorbitol | 0.25 | 0.12 | 1.16 | 0.55 |
| 96 | Octyl sulfobetaine | 0.25 | 0.14 | 1.15 | 0.65 |
| 97 | HEPES | 0.25 | 0.17 | 1.15 | 0.78 |
| 98 | PF-127 | 0.25 | 0.02 | 1.14 | 0.11 |
| 99 | ACES | 0.24 | 0.15 | 1.12 | 0.68 |
| 100 | Maltose | 0.24 | 0.03 | 1.11 | 0.13 |
| 101 | Betaine | 0.24 | 0.03 | 1.11 | 0.16 |
| 102 | Taurine | 0.24 | 0.15 | 1.11 | 0.69 |
| 103 | Sodium Ascorbate | 0.24 | 0.13 | 1.10 | 0.58 |
| 104 | Dextrose Monohydrate | 0.24 | 0.15 | 1.09 | 0.71 |
| 105 | D-(−)-Fructose | 0.23 | 0.11 | 1.09 | 0.50 |
| 106 | D-glucose | 0.23 | 0.22 | 1.08 | 1.01 |
| 107 | Potassium Acetate | 0.23 | 0.05 | 1.07 | 0.23 |
| 108 | Glycine | 0.22 | 0.12 | 1.04 | 0.57 |
| 109 | Trisodium citrate dihydrate | 0.22 | 0.08 | 1.02 | 0.37 |
| 110 | 3-Amino-4-methoxybenzene- | 0.22 | 0.07 | 1.00 | 0.33 |
| | sulfonic acid | | | | |
| 111 | Phosphate buffered saline | 0.22 | 0.09 | 1.00 | 0.41 |
| | (pH 7.4) | | | | |
| 112 | Boric Acid | 0.21 | 0.09 | 1.00 | 0.44 |
| 113 | Ammonium Acetate | 0.20 | 0.08 | 0.95 | 0.35 |
| 114 | L-Cysteine hydrochloride | 0.20 | 0.10 | 0.93 | 0.47 |
| 115 | Creatinine | 0.19 | 0.01 | 0.89 | 0.06 |
| 116 | Polyvinylpyrrolidone | 0.19 | 0.06 | 0.88 | 0.27 |
| 117 | Hydrolyzed Soy Protein | 0.19 | 0.20 | 0.86 | 0.94 |
| | (2000 Da) | | | | |
| 118 | Methylparaben | 0.17 | 0.03 | 0.81 | 0.12 |
| 119 | Hydroxypropyl Cellulose | 0.17 | 0.13 | 0.80 | 0.60 |
| 120 | MOPS | 0.17 | 0.03 | 0.80 | 0.13 |
| 121 | EPPS | 0.17 | 0.16 | 0.79 | 0.74 |
| 122 | Sodium Gluconate | 0.15 | 0.12 | 0.69 | 0.57 |
| 123 | Gentisic Acid | 0.13 | 0.02 | 0.62 | 0.08 |
| 124 | L-Cysteine | 0.13 | 0.04 | 0.60 | 0.20 |
| 125 | Succinic Acid | 0.13 | 0.08 | 0.59 | 0.38 |
| 126 | Gluconolactone | 0.12 | 0.01 | 0.58 | 0.06 |
| 127 | Alpha cyclodextrin | 0.12 | 0.11 | 0.55 | 0.51 |
| 128 | Sodium Bisulfite | 0.11 | 0.02 | 0.53 | 0.08 |
| 129 | Carboxymethylcellulose Sodium | 0.11 | 0.00 | 0.53 | 0.01 |
| 130 | Lauryl sulfobetaine | 0.11 | 0.09 | 0.52 | 0.41 |
| 131 | Tartaric Acid | 0.08 | 0.01 | 0.37 | 0.05 |
| 132 | Malic Acid | 0.08 | 0.01 | 0.35 | 0.06 |
| 133 | Maleic Acid | 0.07 | 0.02 | 0.34 | 0.07 |
| 134 | Glycolic acid | 0.06 | 0.02 | 0.28 | 0.12 |
| 135 | ADA | 0.06 | 0.00 | 0.26 | 0.01 |
| 136 | Citric Acid | 0.04 | 0.01 | 0.20 | 0.03 |
| 137 | Adipic acid | 0.04 | 0.00 | 0.17 | 0.01 |
| 138 | Sodium Sulfite | 0.04 | 0.02 | 0.16 | 0.12 |
| 139 | Sodium Pyrophosphate | 0.03 | 0.01 | 0.13 | 0.04 |
| 140 | Tannic Acid | 0.02 | 0.00 | 0.08 | 0.02 |
| 141 | Carrageenan | 0.00 | 0.00 | 0.01 | 0.01 |

### In Silico and High-Throughput Excipient Screen

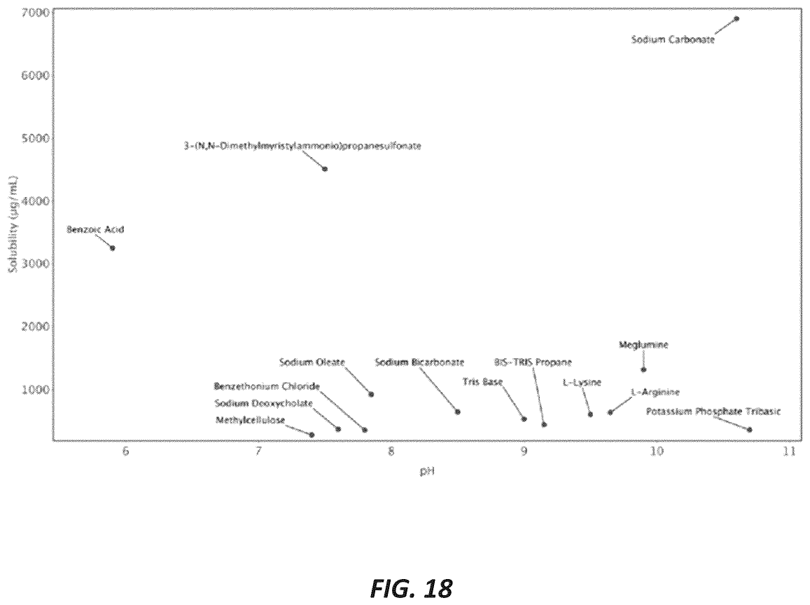

The screening results in FIG. 1A and TABLE 1 help to identify pharmaceutical excipients for solid ReST glucagon formulations that can dissolve rapidly in physiological fluid. The screening identified chemical compounds capable of enhancing the solubility of glucagon in neutral-pH phosphate buffer. See FIG. 18 for additional information related to the solubility of glucagon in excipient solutions. The library of compounds included in the screen include 141 solid (Tₘ>37° C.) and water-soluble (≤1 mg/mL in water) pharmaceutical excipients used in FDA-approved parenteral formulations supplemented with pharmaceutical polymers, surfactants, buffers, and salts used in non-parental formulations.

For the screening, stock solutions of each pharmaceutical excipient were prepared in PBS (0.01 M, pH 7.4) at a concentration of 1 mg/mL. These solutions were transferred to 2 mL polypropylene deep well plates and frozen at −80° C. for storage. 4 mg of lyophilized glucagon powder was dispensed into each well of a 0.75 mL polypropylene Micronic plate followed by the addition of 0.5 mL of excipient stock solution. The plates were sealed and placed on an orbital shaker (50 rpm) at 37° C. for 30 minutes. The supernatant containing solubilized glucagon was separated from the insoluble drug by centrifugation (4000 g, 5 minutes) and filtration (0.22 μm 96-well cellulose acetate filter plate, 1500 g, 5 minutes) prior to analysis. The concentration of solubilized glucagon within the supernatant was quantified using reverse-phased (RP) HPLC.

The concentration of glucagon was analyzed using an Agilent 1260 Infinity I HPLC equipped with a UV detector, and an Agilent Poroshell 120 EC-C-18 2.7 μm 3.0×50 mm column (Agilent 699975-302). Samples were loaded onto the column using a mobile phase including 50% acetonitrile and 50% of 0.1% trifluoroacetic acid. Elution from the column was carried out over a three-minute period with a flow rate of 0.5 mL/min for three minutes starting at 50% acetonitrile and ending at 25% acetonitrile. Glucagon was detected using a UV absorbance of 240 nm, with the primary peak eluting at 2.1 minutes. Glucagon concentration was quantified using the peak area.

### ReST Glucagon Formulations

The pharmaceutical excipients identified by the screening can be used in ReST glucagon formulations that address the longstanding challenge of making a storage-stable glucagon formulation that can be administered directly to the body without prior reconstitution. Previous efforts to develop such a formulation focused on stabilizing the glucagon peptide in dilute aqueous and non-aqueous solutions, or low-density powders. Rather than stabilizing the glucagon peptide, we pursued identifying high-density solid glucagon formulations that rapidly dissolve in neutral pH solutions, such as interstitial fluid, to enable storage and administration as a compact dry solid.

The screening identified ten water-soluble solid excipients as capable of enhancing glucagon dissolution in pH 7.4 PBS. One of these excipients—MSB—can enhance solution glucagon concentration by up to 500% compared to unformulated glucagon and has a pH-independent mechanism of action. A high-density solid formulation composed of 20% MSB and 80% lyophilized glucagon can start dissolving within minutes following incubation in a neutral pH buffer.

Solid ReST glucagon formulations are especially stable during storage. A solid ReST glucagon formulation exhibited little to no chemical and physical degradation following storage at 40° C. for up to 14 days and preserved biological effect following 30-day storage. This dry formulation exhibits exceptionally high drug density, e.g., up to 80% by weight, which enables space efficient loading into volume-constrained drug delivery devices (e.g., injectable implants, ingestible micro-injectors, or transdermal microneedle patches). Administration of high-density ReST pellets into the subcutaneous tissue of diabetic rats elicited rapid peptide dissolution and hyperglycemic effects on timescales that are comparable to subcutaneous injection. High-density ReST formulations administered by next-generation delivery devices may enable new modes of glucagon therapy so long as the kinetics of formulation administration are in line with the application-specific windows of opportunity identified by the CGM analysis.

### Formulation and Dissolution Kinetics of Solid Glucagon Tablets

The excipients were cryo-milled and sieved to a particle size of 63-120 μm (230-120 mesh) before being combined with lyophilized glucagon powder. Solid glucagon formulations were manufactured by tablet compaction of the drug-excipient mixture (1 mm diameter die, 400 N compaction force) using an RD10A Natoli Tablet press. Magnesium stearate, a common lubricant used in tableting, was excluded from this process. The solid formulations were stored at 4° C. until use.

The dissolution kinetics of the solid formulations were assessed by incubating the solid glucagon formulations in 2 mL of pH 7.4 PBS, stored at 37° C. on an orbital shaker. At predetermined timepoints, 200 μL of supernatant was removed for HPLC analysis and an equivalent volume of release medium was replaced. The samples were diluted with equal parts of acetonitrile and analyzed using RP-HPLC.

### Glucagon Formulation Stability

The stability of glucagon formulations was evaluated by packaging the formulations within desiccated and sealed glass vials, followed by incubation at 40° C. and 75% relative humidity for 14 to 30 days. An identical batch of formulations was stored at 4° C. The chemical stability of the formulations following incubation was assessed by measuring glucagon purity using RP HPLC, glucagon structure using circular dichroism, and bioactivity in diabetic rats.

Sample preparation for the chemical purity analysis proceeded by diluting glucagon formulations to a final drug concentration of 250 μg/mL in equimolar acetonitrile and water, and further supplemented with 2 μL of 1M HCl. The chemical purity analysis was performed using HPLC as described above. Glucagon purity was determined as the percent area of the main peak relative to the total area of all peaks within the RP-HPLC chromatogram.

Sample preparation for circular dichroism analysis proceeded by diluting samples to a final concentration of 50 μM in 0.154 mM pH 7.4 PBS. Samples were analyzed using a JASCO circular dichroism using a 2 mm path-length quartz cuvette.

### Transdermal, Mini-Dosing Microneedle Patches for Rapid Mini-Dose Glucagon Delivery

Glucagon mini-dosing can be used as an effective treatment for several hypoglycemic conditions, including in children with impending or mild hypoglycemia, and for prevention of exercise-related hypoglycemia. Glucagon mini-dosing is effective in these use cases with low doses of subcutaneous glucagon, typically in the range of 50 μg to 150 μg. Advantages of glucagon mini-dose therapy include reduced risk of unwanted weight gain that could otherwise occur with frequent rescue carbohydrate treatments, reduced risk of ensuing hyperglycemia following use of glucose tablets to prevent hypoglycemia during exercise, and reduced risk of hypoglycemia unawareness by mitigating repeated episodes of hypoglycemia.

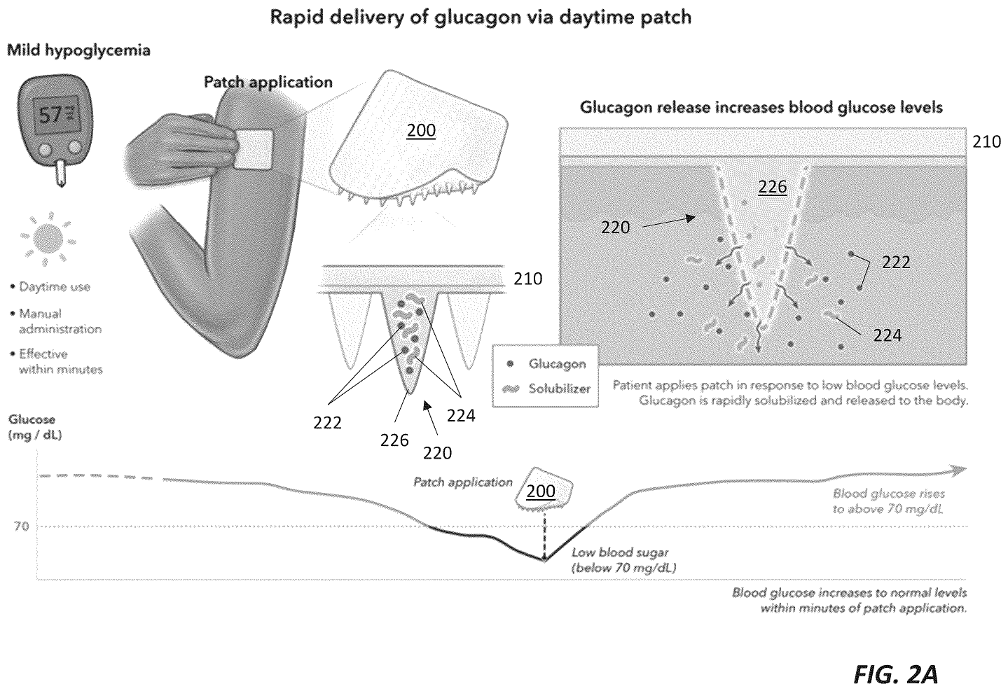

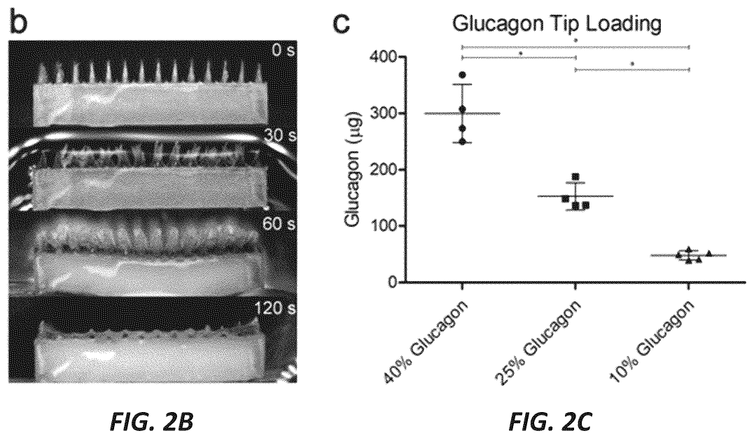

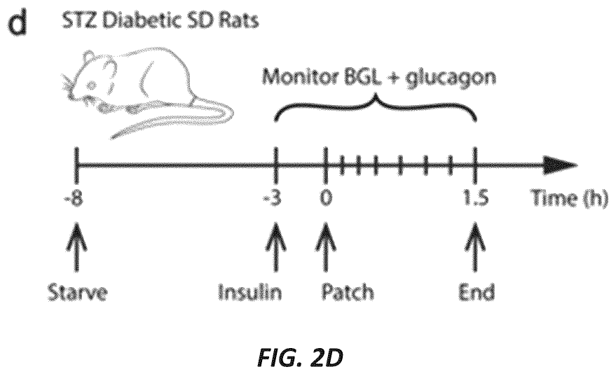

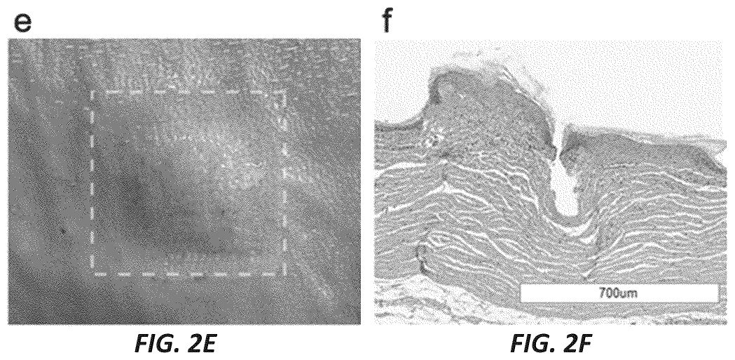

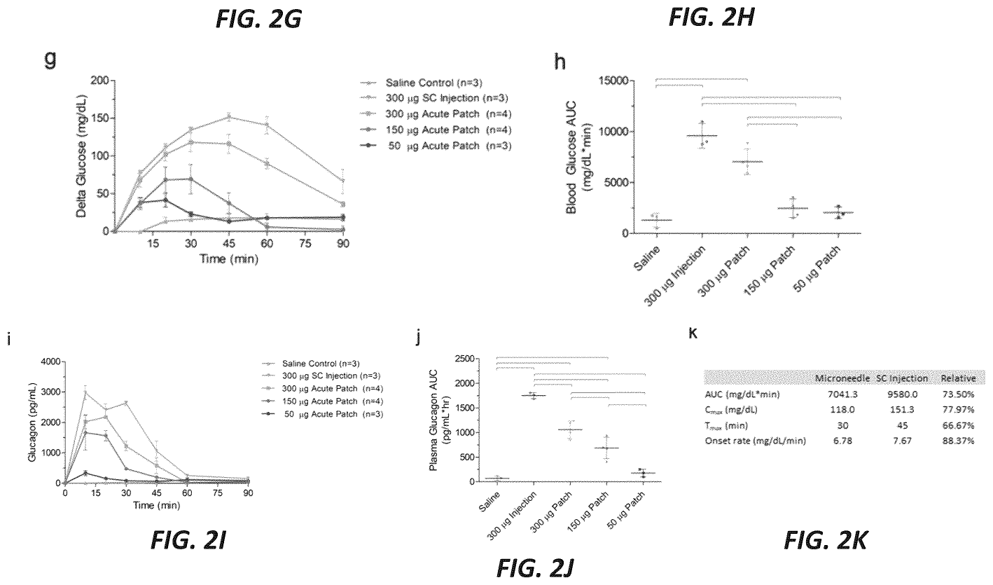

FIGS. 2A-2J illustrate glucagon mini-dose treatment with transdermal, mini-dosing microneedle patches. This treatment is painless, convenient, accurate, and suitable for use in a home setting. It works with a short delay between application and glucagon liberation, dissolution, and transport to the bloodstream.

FIG. 2A shows a transdermal, mini-dosing microneedle patch 200 that measures 1 cm by 1 cm in size and features a 14-by-14 array of drug-loaded microneedles 220 on a suitable flexible, adhesive backing 210, such as an adhesive bandage. Other mini-dosing microneedle patches may be larger or smaller, have different shapes, have arrays with more or fewer microneedles, and/or microneedles of other sizes and pitches. For instance, square mini-dosing microneedle patches can range between 0.5 cm to 4 cm in length/width. Circular mini-dosing microneedle patches can range from 0.5 cm to 4 cm in diameter.

Each microneedle 220 has a height of about 0.5 mm to about 2 mm (e.g., about 0.8 mm to about 1.2 mm, or about 1 mm). Each microneedle 220 is formed of a polymer matrix 226 loaded with a solid ReST glucagon formulation 222 and a solubilizer 224. Suitable polymers for the polymer matrix 226, which is not necessarily sensitive to pH, include natural or synthetic polymers that are (1) hydrophilic, (2) stiff when dry, and (3) compatible with glucagon. The mini-dosing microneedle patch 200 does not include any enzymes (i.e., it does not include glucose oxidase or catalase like the glucose-response microneedle patch described below). Applying the mini-dosing microneedle patch 200 to a person's skin causes the ReST glucagon formulation 222 to be rapidly solubilized and released into the person's body. Glucagon formulations 222 that are more soluble in tissue provide faster drug release kinetics and a faster biological effect.

The mini-dosing microneedle patch 200 may include about 50 μg to about 200 μg of glucagon, glucagon analogue, or glucagon derivative total. A microneedle patch for treating severe hypoglycemia may deliver a total of about 1 mg to 2 mg of glucagon, glucagon analogue, or glucagon derivative. The 48.2 μg±8.1 μg, 152.3 ug±24.7 μg, and 299.8 ug±51.4 μg levels of glucagon loading within the tips of the microneedles 220 shown in FIG. 2C were achieved by adjusting the ratio of drug formulation to polymer matrix, e.g., within a range of 10-50% drug formulation. The drug loading within the microneedle tips is highly reproducible, with variability increasing proportional to the absolute amount of drug loaded. A small (e.g., ˜2%) loss of glucagon purity was observed in the dried transdermal microneedle patches, presumably due to dehydration of the dried transdermal microneedle patches.

FIG. 2F is a photograph of a patch of ex vivo pig skin showing punctures left by a mini-dosing microneedle patch. FIG. 2F shows that construction of the microneedle structures using super-soluble and biocompatible hyaluronic acid and gelatin polymers imparts sufficient rigidity and strength to the microneedles 220 to enable them to penetrate the stratum corneum of ex vivo pig skin while dry. Upon contact with neutral pH solutions (e.g., pH 7.4 phosphate buffered saline), the xerogel microneedles readily imbibed liquid, leading to expansion of microneedle size within 60 seconds, and complete dissolution of both the microneedles and the encapsulated glucagon within 120 seconds as shown in FIG. 2B.

### Transdermal Delivery and Efficacy of Glucagon Mini-Doses in Rats

Painless, self-administrable transdermal microneedle patches can deliver glucagon rapidly enough to prevent onset of mild hypoglycemia and mitigate progression of mild hypoglycemia to severe hypoglycemia. The high-density and solid nature of the ReST glucagon formulation in the microneedles enables (1) facile drug loading into the microneedles, (2) loading of clinically relevant glucagon doses (e.g., 50-150 μg per microneedle, or about 0.2-2.0 mg per patch) within a 1 cm by 1 cm transdermal microneedle patch, and (3) remarkable glucagon stability and preserved biologically active even after storage at 40° C. for 14 days.

Accurate and reproducible control over drug loading within the microneedle structures translated to reproducible dose-dependent drug exposure and hyperglycemic effect in diabetic rats following transdermal patch application. Surprisingly, the solid glucagon formulation delivered by the microneedle patch resulted in a rapid increase in BGL which is comparable to an equivalent dose of liquid glucagon solution injected into the subcutaneous tissue (i.e., significant increase in BGL by 10 minutes and Tₘₐₓ by 30 to 45 minutes).

Transdermal microneedle patches address several challenges that have impeded widespread adoption of glucagon mini-dose treatment of mild hypoglycemia. These challenges include the ability to deliver glucagon (1) in a painless and easy-to-use manner from a compact and portable device, (2) at therapeutically relevant and reproducible doses, and (3) with kinetics that are suitable for prevention of mild hypoglycemia onset in well-controlled and general type 1 diabetes populations and of progression to severe hypoglycemia when administered at the onset of pre-hypoglycemia or mild hypoglycemia.

FIG. 2D illustrates the pharmacokinetics and pharmacodynamics of the glucagon mini-dose microneedle patches in diabetic rats. Transdermal application of the patches loaded with 50 μg, 150 μg, or 300 μg of glucagon resulted in an increase in blood glucose levels detectable within 10 minutes following administration, and a Tₘₐₓ of 30-34 minutes as shown in FIG. 2G. The magnitude of the hyperglycemic effect, determined by change in blood glucose levels and area under the blood glucose-time curve (AUC_{BG}), was found to increase in a dose-dependent manner as shown in FIG. 2H. The hyperglycemic effects observed in the treated rats were further accompanied by a similar dose-dependent increase in plasma glucagon levels as shown in FIGS. 21 and 2J. While transdermal delivery of 300 μg of glucagon from the microneedle patch resulted in similar pharmacokinetics and onset of biological activity compared to treatment with an equivalent dose of glucagon administered via subcutaneous injection, the total bioavailability and bioactivity of the patches were reduced at 73% and 60%, respectively, as shown in FIG. 2K. This reduction is likely due to a combination of incomplete systemic absorption of the drug in the dermal tissue, and a small reduction (e.g., ˜2%) in drug stability in the microneedle dosage form. Beneficially, the amount of glucagon delivered by the patches and the resulting hyperglycemic effect was found to be highly reproducible.

Transdermal application of the microneedle patches to the skin appears to be well-tolerated, with no signs of redness or permanent damage to the skin surface as shown in FIG. 2E. The histological analysis of the application site shown in FIG. 2F revealed successful penetration of the stratum corneum, and no signs of swelling, irritation, or inflammation. Taken together, these results suggest that the glucagon mini-dose microneedle patches can pierce the skin of the diabetic rats to access the underlying interstitial fluid leading to accurate, dose-dependent, and rapid systemic delivery of glucagon that is aligned with the timing needs to prevent either onset of mild hypoglycemia (e.g., 44 to 60 minutes) or progression of mild-to-severe hypoglycemia (e.g., 16 to 25 minutes) in type 1 diabetes patients during the day and at night.

### Fabrication of Mini-Dosing Microneedle Patches

The mini-dosing microneedle patches shown in FIG. 2B and tested here were molded in polydimethylsiloxane (PDMS) microneedle molds prepared by casting a steel microneedle master within a well-mixed solution of PDMS (1:10 ratio of crosslinker to base). The PDMS molds were heated at 60° C. for 12 hours followed by the removal of the steel microneedle master, then filled with a combined polymer stock and glucagon solution. The polymer stock solution was prepared by dissolving 3 mg of hyaluronic acid and 3 mg of gelatin in 80 μL of 0.35 M HCl and incubating the resulting solution at 37° C. for 4 hours. In the examples here, the glucagon solution was prepared by dissolving 1, 3, or 6 mg (10%, 25%, or 40% drug loading, respectively) of lyophilized human recombinant glucagon and 3 mg of MSB in 40 μL of 0.35 M HCl. The polymer stock and glucagon solutions were then combined, mixed thoroughly, and centrifuged to remove air bubbles. The mixture was then dispensed into the PDMS microneedle mold and centrifuged at 4000 rpm for 3 minutes. The microneedles 220 were left at room temperature to dry overnight. 40 μL of medical-grade epoxy was introduced to the PDMS microneedle mold and centrifuged at 500 rpm for 1 minute. The epoxy was allowed to solidify overnight, providing a backing for the microneedles 220. Other materials, such as thick, stiff, bendable polymer sheet or a stiff piece of plastic tape, can be used as backings instead of epoxy. The demolded microneedles 220 were attached to an adhesive bandage (flexible, adhesive backing 210) and stored in a desiccated container at 4° C. until use.

### Glucose-Responsive Microneedle Patches for Hypoglycemia-Triggered Glucagon Delivery

Automated hypoglycemia-triggered glucagon delivery may mitigate the risk of hypoglycemia and eliminate the burden of persistent blood glucose monitoring and manual intervention for people with diabetes and their caregivers. Hypoglycemia-triggered glucagon delivery is particularly useful for detecting and treating people with diabetes who are asleep. Unfortunately, chemically driven glucose-responsive glucagon delivery systems proposed to date share design features that may impede clinical translation, including (1) use of a non-specific hypoglycemia-triggering mechanism based on phenylboronic acid, which interacts with interfering sugars and compounds commonly found in the body (e.g., fructose, galactose, mannose, sialic acid), and (2) glucagon loading strategies that are prone to continuous drug elution at euglycemic and even hyperglycemic conditions, which may exacerbate hyperglycemia and lead to hyperglucagonemia.

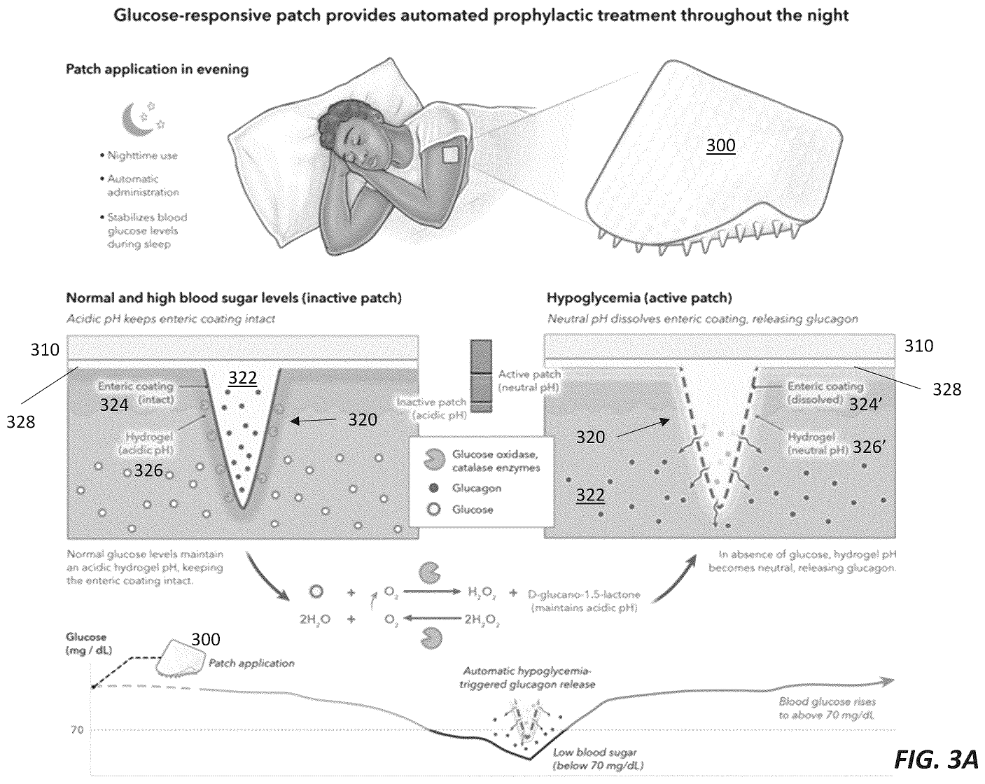

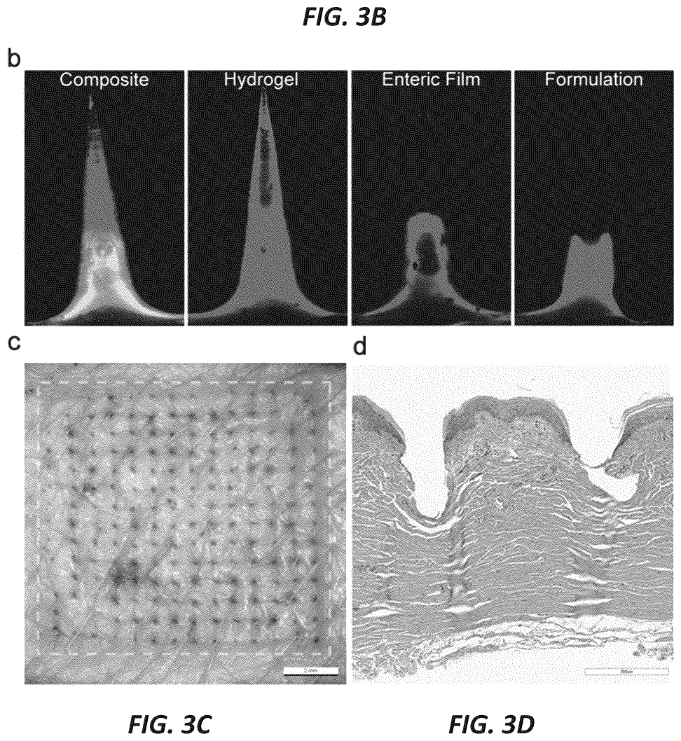

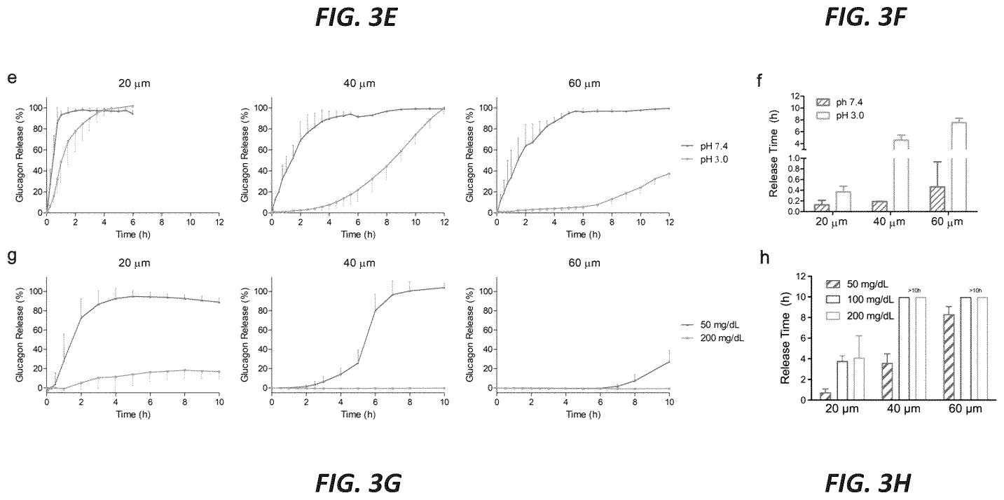

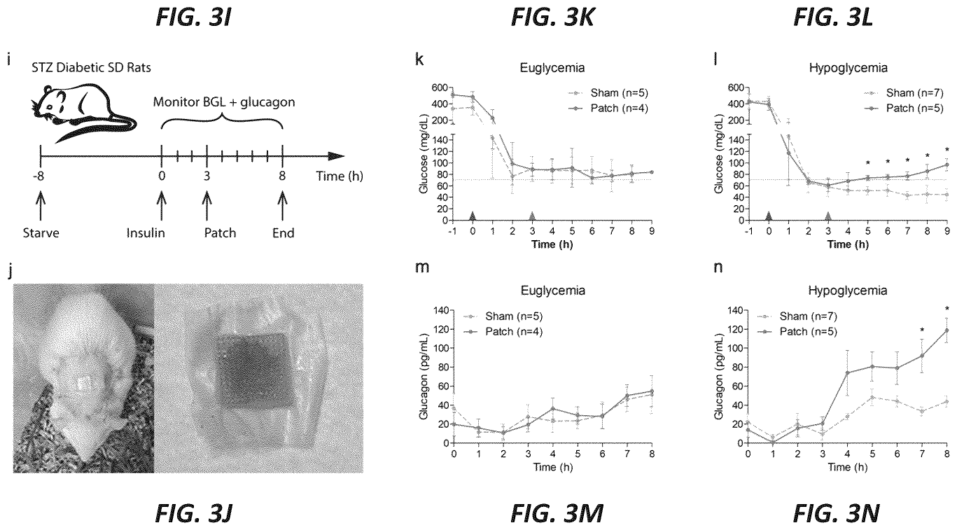

FIGS. 3A-3N illustrate a prophylactic, transdermal, glucose-responsive microneedle patch 300 that provides automated and persistent glucagon delivery during episodes of nocturnal hypoglycemia without the drawbacks of other glucagon delivery systems. This glucose-responsive microneedle patch 300 leverages a hypoglycemia-responsive glucagon-release mechanism based on the glucose oxidase enzyme, which is highly specific for glucose, and exhibits no drug release prior to hypoglycemia triggering. In other words, the hypoglycemia-responsive glucagon-release mechanism prevents both premature elution of glucagon to the body (even at low levels) during periods of euglycemia or hypoglycemia and accidental triggering by non-specific analytes.

The glucose-responsive microneedle patch 300 can be used at night as a prophylactic treatment for nocturnal hypoglycemia: it can be safely applied before sleep, worn throughout the night to protect against nocturnal hypoglycemia, and discarded in the morning. It can also be used in anticipation of a hypoglycemia event or prior to operation of a motor vehicle to ensure that hypoglycemia does not occur during the operation of the motor vehicle. Other use cases include preventing hypoglycemia during intensive insulin therapy where the risk of hypoglycemia is relatively high compared to insulin therapy with relaxed glycemic targets.

FIG. 3A shows a detailed view of the prophylactic, transdermal, glucose-responsive microneedle patch 300 and its use. The glucose-responsive transdermal microneedle patch 300 includes an array of microneedles 320 on a flexible adhesive back 310, such as a flexible adhesive bandage. Each microneedle 320 is comprised of four distinct layers: (1) a crosslinked hydrogel matrix 326 containing glucose oxidase and catalase or MnO₂ enzymes or analogues of these enzymes or similar enzymes, (2) a solid ReST solid glucagon formulation 322 (e.g., containing glucagon, a glucagon analog, or a glucagon derivative and MSB or another suitable excipient), (3) a conformal enteric polymer film or enteric coating 324 that acts as a pH-sensitive barrier between the hydrogel 326 and the solid ReST glucagon formulation 322, and (4) a solid epoxy backing 328 that provides structural support to the glucose-responsive microneedle patch 300. The solid nature of the ReST glucagon formulation 322 facilitates high drug stability during storage, structural integrity during transdermal application, and timely drug delivery due to its rapid and enhanced solubility in neutral pH solutions.

The glucose-responsive transdermal microneedle patch 300 leverages the ReST glucagon formulation 322 to achieve high drug loading and compact patch size. Suitable glucagon-to-excipient ratios can be wide (e.g., 10% to 90%). A suitable excipient is an excipient that facilitates dissolution of glucagon in neutral or near-neutral pH (e.g., pH 6 to 7.4) without making the solution acidic (the enteric polymer may be stabilized by a formulation that is acidic in pH). Suitable excipients include sodium carbonate, meglumine, sodium phosphate tribasic, myristyl sulfobetaine, kollidon HS 15, dodecylphosphocholine, 1-glutamine, and PIPES. Other suitable excipients may include excipients that dissolve glucagon in basic solutions (e.g., sodium carbonate or sodium phosphate tribasic).

The solid nature of the ReST glucagon formulation 322 facilitates easy integration with the microneedles 320, preserves rigidity of the dry xerogel microneedles 320, and circumvents the challenge of stabilizing liquid glucagon formulations during long-term storage. The rapid and pH-independent dissolution of the ReST glucagon formulation 322 in physiological fluids, including interstitial fluid wicked into the swollen hydrogel microneedles 320 following transdermal application, enables the timely release of glucagon from the transdermal microneedle patch 300 following exposure to low glucose levels. Additionally, the patch, in some embodiments, does not comprise instrumentation or on-board power support thereby providing an inexpensive and easy-to-deliver platform to manage a patient's hypoglycemia.

The glucose-responsive drug release kinetics (or lack thereof) from the glucagon-response transdermal microneedle patch 300 in hypoglycemic and euglycemic conditions depend on the pH-responsive dissolution behavior of the enteric film barrier 324. The enteric polymer film 324 is formulated to remain insoluble in acidic pH environments below the material's pKₐ and to become soluble in more neutral pH environments; however, the enteric polymer film 324 may not dissolve instantaneously at neutral pH and may not remain indefinitely insoluble at low pH. Put differently, the enteric polymer film 324 that encapsulates the glucagon 322 is a pH-responsive material that is relatively soluble or swellable in neutral pH and relatively insoluble or non-swollen in acidic pH.

For example, the enteric polymer film 324 can be made of shellac, which has a large pH sensitivity (i.e., the ratio between release time at pH 5.0 and pH 7.4) compared to other enteric polymer systems. Incorporating a 20-micron thick shellac barrier within the microneedle patch 300 yielded an average response time of 45.5 minutes, which falls within the window of opportunity to provide automated glucagon delivery following the onset of nocturnal hypoglycemia in type 1 diabetes patients.

The composition and thickness of the enteric polymer film 324 (including pKₐ and molecular weight) affect the kinetics of glucagon release from the microneedles 320. For instance, the enteric polymer film 324 can be 10-400 microns thick, with thicker enteric polymer films providing slower glucagon release rates in both low and neutral pH environments. The composition and thickness of the enteric polymer film 324 can be selected to achieve the desired glucose-responsive drug delivery profiles for the prophylactic treatment of nocturnal hypoglycemia. Suitable compositions for the enteric polymer films 324 include shellac, as described above, as well as methyl acrylate-methacrylic acid copolymers, cellulose acetate phthalate (CAP), cellulose acetate succinate, hydroxypropyl methyl cellulose phthalate (HP-50, HP-55, HP55S), hydroxypropyl methyl cellulose acetate succinate (hypromellose acetate succinate LF grade, MG grade, HF grade), polyvinyl acetate phthalate (PVAP), methyl methacrylate-methacrylic acid copolymers (Eudragit L100-55, L30D-55, L-100, L12,5, FS30D), cellulose acetate trimellitate, and zein. Other enteric materials with better pH-sensitive dissolution profiles may provide faster delivery kinetics for other applications.

Likewise, alternative microneedle patch architectures, including layer-by-layer designs and encapsulation of enterically coated solid glucagon microparticles, may facilitate glucagon rescue from multiple nocturnal hypoglycemic events. The microneedles shown in FIGS. 3A and 3B have three layers: an outer hydrogel layer with enzymes (H), a middle enteric polymer layer (E), and an inner layer of glucagon formulation (the core) (G). Microneedles, microparticles, or other implants may have more layers, e.g., five layers (H, E, G, E, G), seven layers (H, E, G, E, G, E, G), and so on. For instance, a glucagon microparticle may include a glucagon core coated with an enteric polymer layer, which is coated in turn with an outer hydrogel layer loaded with glucose oxidase and catalase or MnO₂. The glucagon core can have a diameter that ranges from 5 microns to 200 microns, and the enteric polymer film can be from 5 microns to 180 microns thick. The number of microparticles depends on the size(s) of the microparticles and the desired dose, among other things. Considering the high prevalence (i.e., 20% to 30%) of parent hypoglycemic events with one or more follow-on events, a wearable system that provides persistent elution of glucagon following hypoglycemic triggering may be desirable.

FIG. 3A also illustrates the glucose-responsive mechanism that enables the hypoglycemia-triggered release of glucagon from the glucose-responsive microneedle patch 300. The glucose-responsive microneedle patch 300 is intended to be applied during periods of euglycemia, that is, when the subject's blood sugar levels are normal or high, as shown at lower left in FIG. 3A. Under these conditions, the normal glucose levels help keep the hydrogel layer's pH acidic, which in turn keeps the enteric coating 324 intact, as shown at middle left in FIG. 3A, and prevents the microneedles 320 from releasing glucagon 322. When glucose levels fall, the hydrogel layer 326′ becomes neutral, causing the enteric coating 324′ to dissolve and releasing the ReST glucagon formulation 322 from the microneedle 320 as shown at right in FIG. 3A.

This glucose-responsive mechanism is driven by two enzymatic reactions catalyzed by the glucose oxidase and catalase enzymes embedded in the hydrogel layer 326. These reactions are shown at the center of FIG. 3A: (1) the glucose oxidase catalyzes the oxidation of β-d-glucose into D-glucono-1,5-lactone and hydrogen peroxide, and (2) the catalase breaks down the hydrogen peroxide byproduct into water and a regenerated oxygen molecule, which propagates the enzymatic reactions. The final D-glucono-1,5-lactone product is rapidly hydrolyzed into gluconic acid leading to a drop in pH in the local microenvironment. An abundance of glucose during periods of euglycemia and hyperglycemia drives this pair of enzymatic reactions forward, resulting in ample production of gluconic acid and maintenance of an acidic microenvironment within the hydrogel matrix. This low pH environment inhibits the dissolution of the enteric polymer film, thereby preventing glucagon release from the glucose-responsive microneedle patch 300. Conversely, the diminished interstitial glucose concentration during periods of hypoglycemia decreases the production of gluconic acid, leading to pH neutralization within the hydrogel 326, rapid dissolution of the pH-sensitive enteric polymer film 324, and release of the glucagon 322 from the microneedle 320.

Alternative designs could use glucose dehydrogenase with a suitable cofactor to sense glucose levels and trigger release of glucagon instead of glucose oxidase and catalase.

FIG. 3B shows confocal fluorescence microscopy images of the microneedle patch. These images reveal the distinct hydrogel (left two images), enteric polymer film (middle right image), and glucagon formulation (right image) layers within the microneedle. FIG. 3C shows the ability of the microneedles to penetrate the skin. The dark dots in FIG. 3C were left by methylene blue solution applied to ex vivo pig skin following transdermal application and removal of a transdermal microneedle path. The visible array of dark dots reveals the locations of microneedle penetration through the stratum corneum and localized dye uptake into the pig skin. The histological analysis in FIG. 3D shows microneedle penetration through the stratum corneum, epidermis and into the subdermal layers of the skin.

To confirm the pH-mediated glucose-responsive drug release mechanism, patches constructed using 20 μm, 40 μm and 60 μm thick shellac enteric polymer films were incubated in buffered acidic or neutral-pH release medium at 37° C. under agitation to simulate in vivo euglycemic and hypoglycemic conditions, respectively. Drug release kinetics were determined by measurement of glucagon concentrations in the supernatant over a 12-hour period as shown in FIG. 3E. The release time of each patch, quantified as the time required to achieve 10% of total drug release, was determined for both acidic and neutral pH release conditions, and the ratio was used to calculate the device's responsiveness (R=T_{pH 3.0}/T_{pH 7.4}), shown in FIG. 3F). The glucose-responsive microneedle patches incubated in pH 7.4 release medium (simulated hypoglycemia) exhibited rapid glucagon release in as little as 10 minutes. In contrast, the glucose-responsive microneedle patches exhibited significantly longer release times (i.e., 0.5, 4, and 7 hours for 20 μm, 40 μm, and 60 μm devices, respectively) when incubated in pH 3.0 (simulated euglycemia) conditions; 3-fold, 24-fold, and 42-fold longer releases, respectively, in acidic pH conditions compared to neutral pH conditions. These results confirm the pH-sensitive drug release mechanism of this patch system and illustrate how the enteric polymer film thickness can be used to control drug release times.

FIGS. 3G and 3H illustrate simulated in vitro glucose-responsive glucagon release kinetics. The glucose-responsive microneedle patches were incubated in pH 7.4 phosphate-buffered saline supplemented with 50 mg/dL or 200 mg/dL glucose at 37° C. under agitation to simulate in vivo hypoglycemic and euglycemic conditions, respectively. Drug release kinetics was determined by measurement of glucagon concentrations in the supernatant over a 10-hour period as shown in FIGS. 3G and 3H. As predicted by the pH-responsive behavior of the system, glucagon release times were faster in low glucose conditions (50 mg/dL) than in high glucose conditions (200 mg/dL) and depended on the enteric polymer chemistry and film thickness.

Glucose-responsive microneedles patches with a 20 μm thick enteric polymer film exhibited rapid glucagon release within 30 minutes following incubation in low glucose conditions, and gradual glucagon release starting at 2 hours in high glucose conditions. Increasing the enteric polymer film thickness to 40 μm and 60 μm progressively delayed glucagon release times in low glucose conditions to 2 and 4 hours while completely suppressing glucagon release for at least 10 hours. These results suggest that an enteric polymer film thickness between 20 μm and 40 μm may be most suitable to minimize glucagon delivery over the course of the night under euglycemic conditions while providing for an acceptable glucagon release kinetic during hypoglycemic conditions in line with patient needs.

### Hypoglycemia-Triggered Glucagon Delivery in Diabetic Rats

FIGS. 3I-3N illustrate evaluation of hypoglycemia-triggered glucagon delivery from glucose-responsive microneedle patches in diabetic rats. The rats were fasted for 8 hours and subsequently treated with recombinant human insulin and ultra-long-acting Insulin Degludec® to induce prolonged euglycemia or hypoglycemia as shown in FIG. 3I. FIGS. 3K and 3L show that stable euglycemia or hypoglycemia was achieved two hours after insulin treatment and persisted out to nine hours following administration. Transdermal, glucose-responsive microneedle patches were applied to the skin three hours after insulin administration and secured in place for the duration of the experiment.

FIG. 3K shows that application of the glucose-responsive microneedle patches to euglycemic rats had no significant effect on blood glucose levels or plasma glucagon levels compared to euglycemic animals treated with sham devices. Likewise, FIG. 3M shows that blood glucose levels remained near-constant between 70 mg/dL and 120 mg/dL both before and after glucose-responsive microneedle patch application and plasma glucagon levels of both patch- and sham-treated euglycemic animals gradually increased from −20 mg/dL to −50 mg/dL over the course of the experiment.

FIG. 3L shows that application of the glucose-responsive microneedle patches to hypoglycemic rats induced an immediate and sustained increase in blood glucose levels resulting in a return to euglycemia within 1-2 hours after patch application. The kinetics of glucagon release were aligned with the desired timing for automated treatment of nocturnal severe hypoglycemia. The blood glucose levels of these animals remained within the euglycemic range for the remainder of the experiment without inducing hyperglycemia (>140 mg/dL). FIG. 3N shows that this rise in blood glucose levels following patch application was accompanied by a significant elevation in plasma glucagon levels. Blood glucose levels of hypoglycemic animals treated with sham devices remained within the hypoglycemic range and continued to gradually decline for the remainder of the experiment. Plasma glucagon levels in this experimental group were comparable to those observed in the patch and sham-treated euglycemic animals. It should be noted that measured glucagon levels are derived from both endogenous production and exogenous sources (i.e., the patch) as the glucagon sequence of both are substantially identical.

### Fabrication of Glucose-Responsive Microneedle Patches

The glucose-responsive microneedle patches tested here were constructed using the following sequential solution casting and spray deposition process. A stock photo-initiator solution was prepared by dissolving 60 mg of Irgacure 12959 in 3.6 mL of DI water and heated at 65° C. for 12 hours while protected from light. A polymer stock solution was prepared by dissolving 100 mg of MeHA, 100 mg of GelMA, and 4 mg of MBA in 1.6 mL of pH 7.4 PBS. This solution was heated at 37° C. for 8 hours to ensure complete polymer dissolution. To this stock, 40 mg of glucose oxidase, 13.3 mg of catalase, and 2.4 mL of photo-initiator stock were added, vortexed, and incubated at 37° C. for 15 minutes. 4 μL of 25% glutaraldehyde solution was added to the solution and vortexed. The homogenous solution was then centrifuged at 1000 rpm for 3 minutes to remove air bubbles.

Next, 80 μL of polymer stock solution was dispensed into each microneedle mold and centrifuged at 4000 rpm for 3 minutes. The molds were protected from light and allowed to dry overnight at room temperature. The dried polymer structure was subsequently exposed to ultraviolet (UV) irradiation in a UV oven for 15 minutes to facilitate UV-initiated hydrogel crosslinking. Preparation of devices for confocal imaging included 4 mg of 3-5 kDa FITC dextran within the formulation.

A 5% w/w shellac stock solution was prepared by dissolving 60 g Protect ENRX in 276 g of isopropyl alcohol. This solution was applied conformally to the back of the crosslinked hydrogel by spray coating. The enteric film was protected from light and allowed to dry overnight at room temperature. 0.5 mg of Texas red NHS ester was incorporated into the shellac film for devices used for confocal imaging.

A glucagon stock solution was prepared by dissolving 10 mg MSB, 10 mg glucagon, 25 mg sorbitol, and 5 mg FITC-dextran in 40 μL of 0.35 M HCl. (Sorbitol was incorporated into the ReST glucagon formulation to increase solution viscosity and facilitate loading into the glucose-responsive microneedle patches and did not lead to a loss in glucagon purity.) 7.6 μL of the glucagon stock solution was dispensed into each mold and allowed to dry at room temperature overnight in the dark. For devices used in confocal imaging, FITC-dextran was replaced with an equivalent mass of sorbitol and 0.5 mg of Cy5.5-labeled glucagon was incorporated into the formulation for each device.

Finally, 40 μL of medical-grade epoxy (Loctite M-21HP) was introduced to the PDMS microneedle mold, centrifuged at 500 rpm for 1 minute, and allowed to cure overnight at room temperature. The microneedles were demolded and attached to an adhesive bandage (flexible, adhesive backing) and stored until use.

### Stability of Glucagon Formulation in Glucose-Responsive Microneedle Patches

The stability of the ReST solid glucagon formulation was evaluated after loading into the glucose-responsive microneedle patches. Incorporation of the formulation into the responsive microneedle patches via solution casting resulted in a <1% loss in glucagon purity while wet (1 and 72 hours), followed by a ˜2% loss in drug purity after complete drying. No further change in the chemical purity of the loaded glucagon was observed after a 14-day incubation of the responsive microneedle patches at 40° C. and 75% relative humidity.

### Enteric-Coated, Solid Glucagon Implants

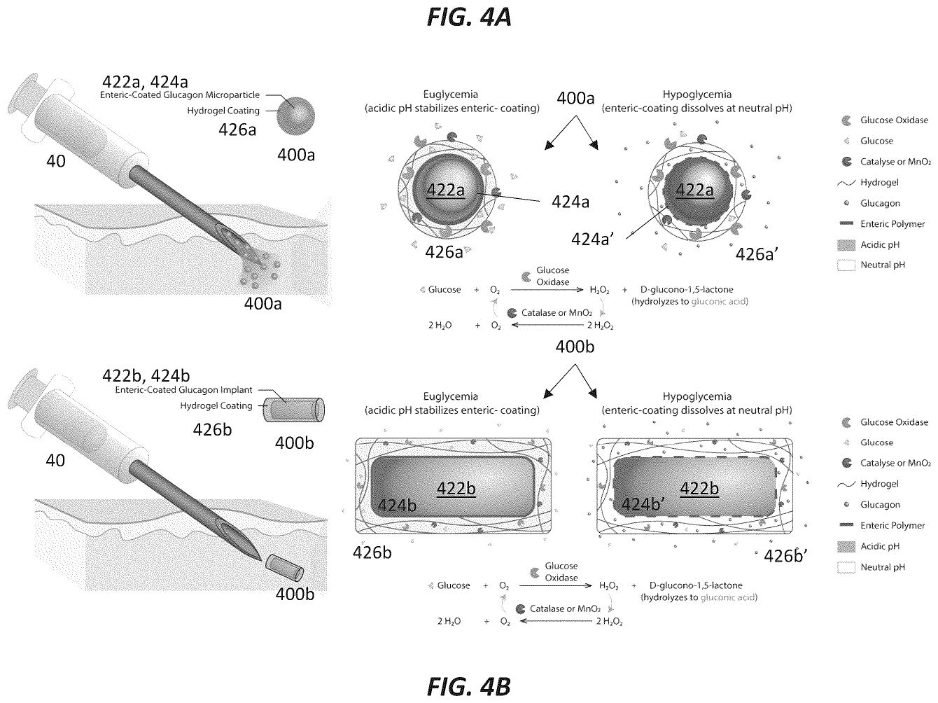

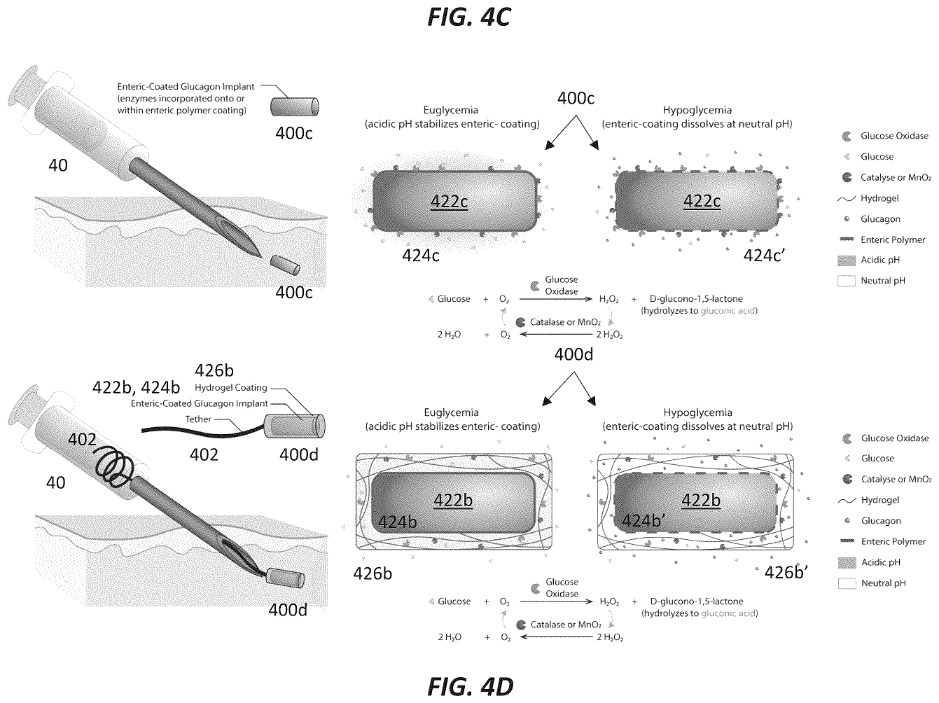

FIGS. 4A-4D illustrate alternatives to microneedle patches for delivering glucagon to people with diabetes: glucose-responsive glucagon implants 400a-400d with solid ReST glucagon formulations 422a-422c coated with pH-sensitive enteric polymer coatings 424a-424c, and, in some cases, hydrogel 426a or 426b. Glucose oxidase and catalase or MnO₂ are incorporated into the enteric polymer coating 424c or the hydrogel 426a or 426b. The glucose-responsive glucagon implants 400a-400d can be injected subcutaneously using an appropriate hypodermic needle or trocar 40, e.g., during a routine doctor's appointment or even at home.

The implants 400a in FIG. 4A are microparticles formed of a solid ReST glucagon formulation 422a. Each implant/microparticle 400a has an inner pH-sensitive enteric polymer film coating 424a and an outer hydrogel coating 426a that is loaded with glucose oxidase and either catalase or MnO₂. The implant 400b in FIG. 4B is formed of a solid ReST glucagon formulation 422b and coated with a pH-sensitive enteric polymer film coating 424b, which in turn is coated with a hydrogel coating 426b that is loaded with glucose oxidase and either catalase or MnO₂. The implant 400c in FIG. 4B is also formed of a solid ReST glucagon formulation 422b but is coated with a pH-sensitive enteric polymer film coating 424b that incorporates glucose oxidase and either catalase or MnO₂. And the implant 400d in FIG. 4D also has a solid ReST glucagon formulation 422b, an inner pH-sensitive enteric polymer film coating 424b, and outer hydrogel coating 426b but is attached to a tether 402 that can be secured to a point outside the person's skin for easier removal or re-positioning.

The implants 400a-400d can be sized to provide one days to several days or possibly several weeks of treatment. The implants 400a-400d can address multiple hypoglycemic episodes, e.g., from one to five hypoglycemic episodes.

The implants 400a-400d in FIGS. 4A-4D feature the same glucose-response mechanism used by the glucose-responsive microneedle patch in FIG. 3A. When the implants 400a-400d are in tissue with a normal or high blood sugar level, the glucose oxidase and catalase or MnO₂ catalyze a pair of enzymatic reactions that produce gluconic acid, causing the environment surrounding the enteric barrier films 424a-424c to remain acidic. So long as the environment remains acidic, the enteric barrier films 424a-424c remains intact, preventing the ReST glucagon formulations 422a-422c from dissolving. When the blood sugar levels fall, the enzymatic reactions produce less gluconic acid causing the environment surrounding the enteric barrier films 424a-424c to become neutral and the enteric barrier films 424a-424c to swell and/or dissolve. As the enteric barrier films 424a-424c swell and/or dissolve, the ReST glucagon formulations 422a-422c dissolve, releasing glucagon into the bloodstream and treating the hypoglycemia.

### Results and Analysis of Continuous Glucose Monitor Data (CGM)

Throughout the disclosure below, results and analysis of the kinetics of hypoglycemic events is described based on CGM data and indicates a set of engineering design principles, such as the boundary conditions around hypoglycemia, that may, in some embodiments, help guide the translational development of situation-specific hypoglycemic interventions.

### Trajectories of Hypoglycemic Events in TID

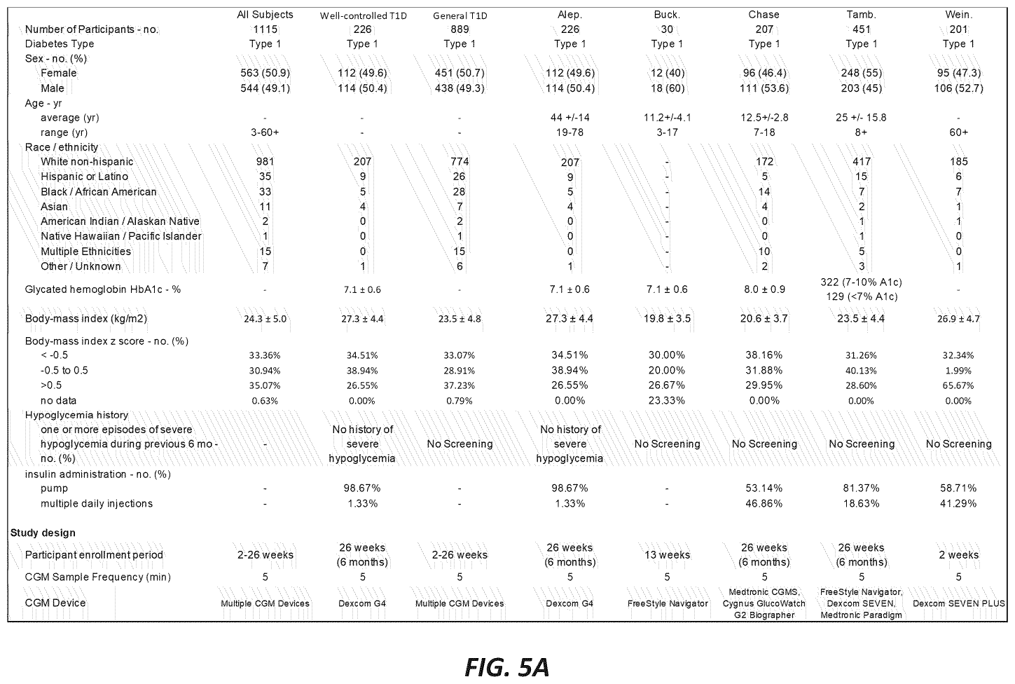

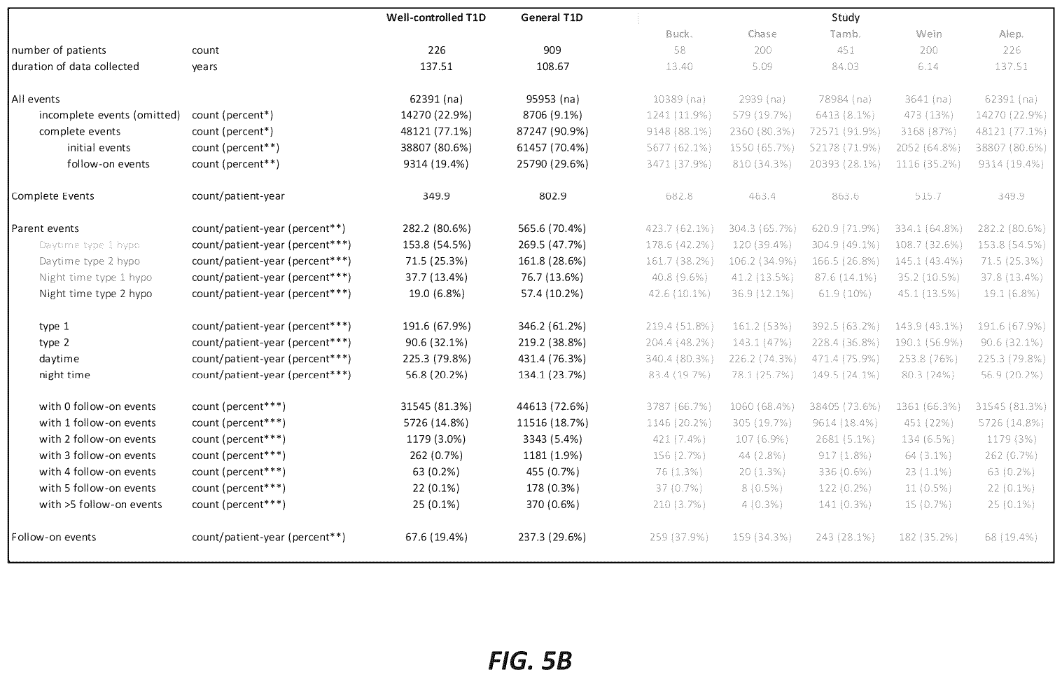

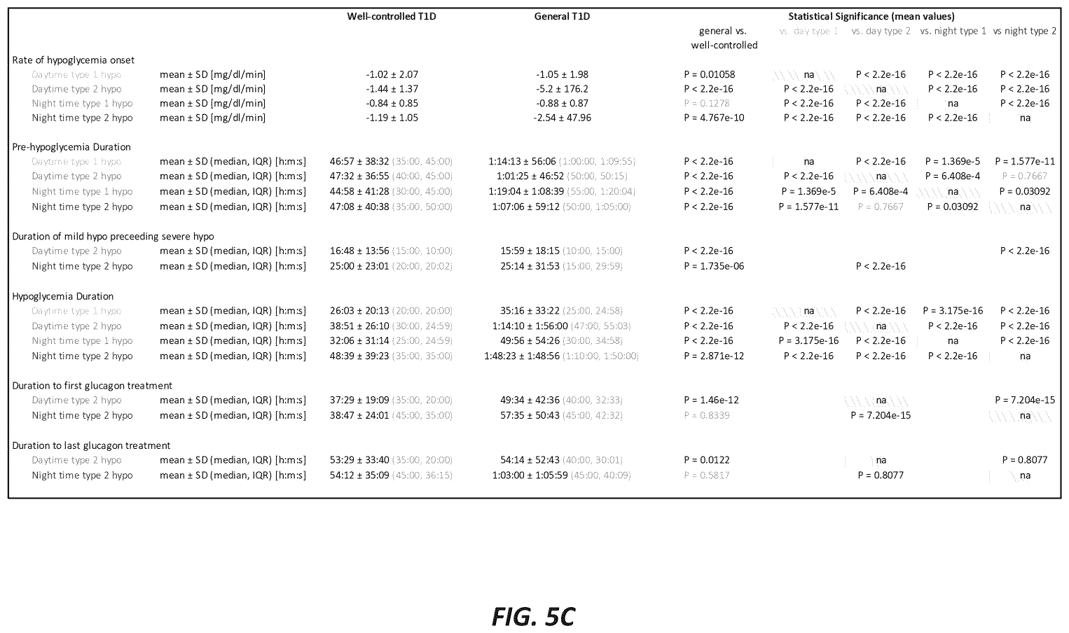

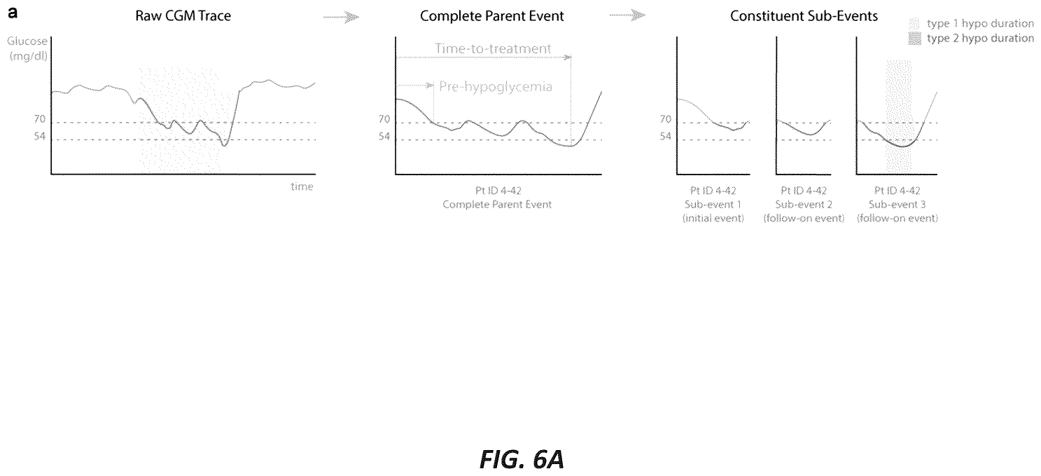

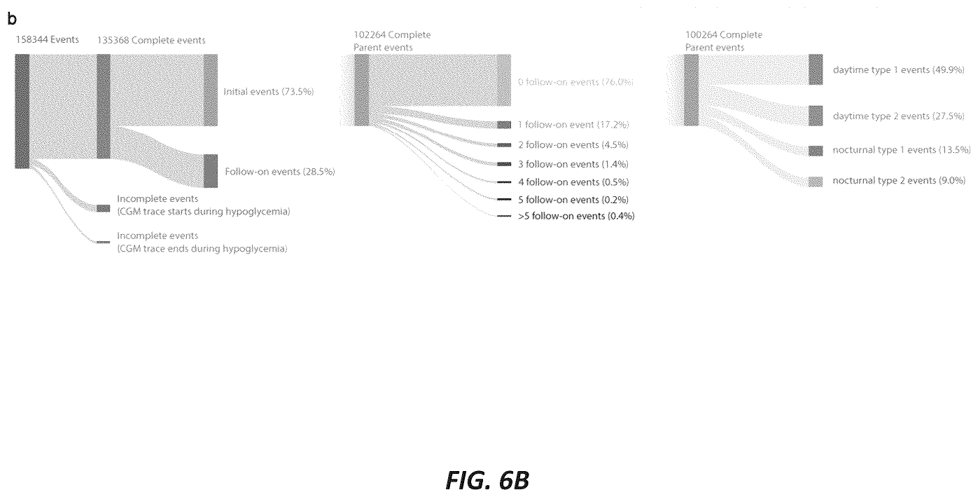

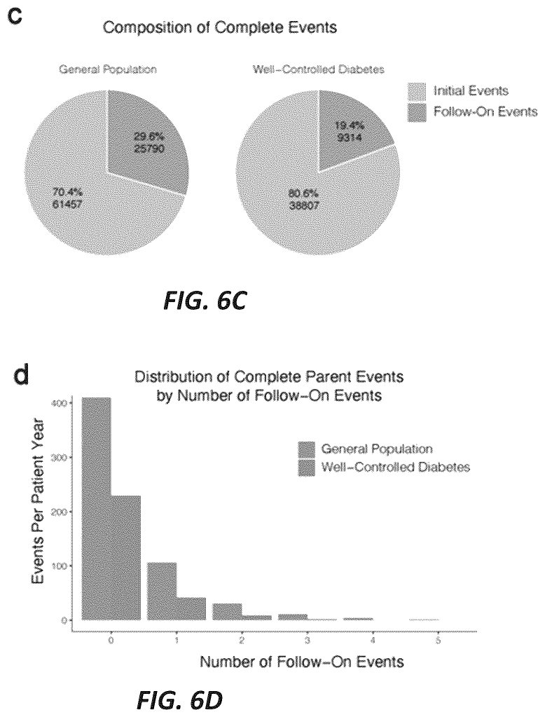

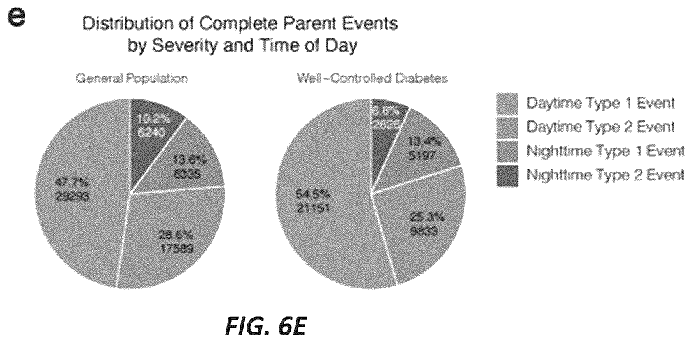

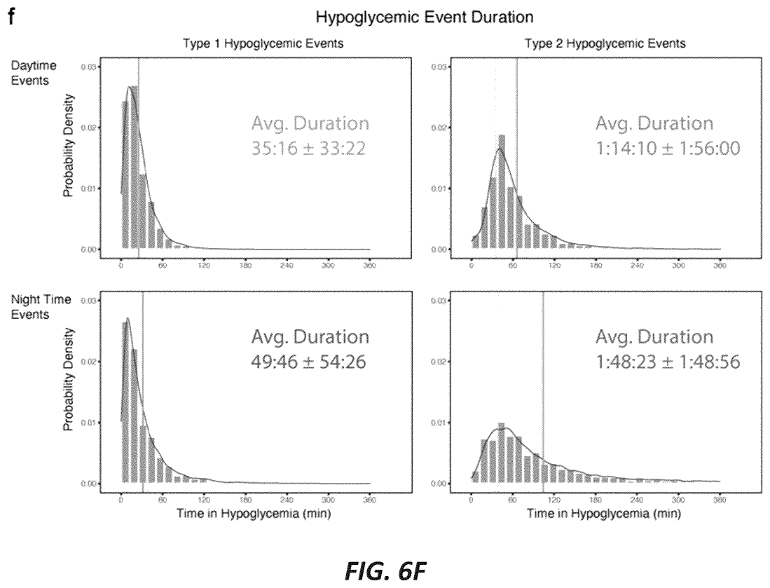

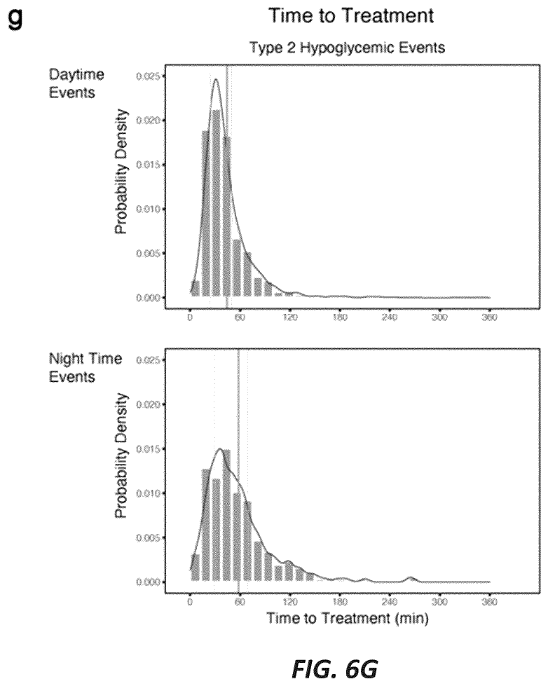

A retrospective analysis of CGM data was performed to identify the trajectories of hypoglycemic events. The aggregated dataset is comprised of 246.18 patient-years of CGM data from 5 publicly available clinical trial datasets and included 1135 subjects (FIG. 5A). Based on patient enrollment criteria, 909 subjects from 4 studies were used to represent the general type 1 diabetes (TID) population while the remaining 226 subjects were considered representative of T1D patients with well-controlled diabetes with no recent history of hypoglycemia. A total of 158344 distinct hypoglycemic events were identified in the dataset, each represented by a CGM trace comprised of a pre-hypoglycemic period and a hypoglycemic period during which blood glucose levels remain below 70 mg/dL (FIG. 6a).

### Incidence Rate

Complete hypoglycemic events were relatively common among the general and well-controlled T1D patient populations with incidence rates of 803 and 350 events per patient-year respectively (FIG. 5B). Independent hypoglycemic events are a relatively clinically relevant metric of incidence given that follow-on events are likely a result of inadequate treatment of a prior event. Therefore, a more holistic approach was adopted in this analysis by associating any follow-on events with the independent hypoglycemic events that closely precede them. Thus, the incidence rate of parent events in the general population was 566 events per patient-year (70.4% of all complete events), and 282 events per patient-year in the well-controlled diabetes population (80.6% of all complete events) (FIGS. 6C-6D). Patients with well-controlled T1D experience 43.6% to 49.9% fewer parent events compared to the general T1D population and ˜10% fewer follow-on events. A surprisingly high proportion (i.e., 24.0%) of parent events are associated with one or more follow-on events indicating that under-treatment of hypoglycemia is common (FIG. 6b, 6d).

Segmentation of parent hypoglycemic events by severity shows that 61-68% of these instances are level 1 hypoglycemia (between 54 and 70 mg/dL), with the remaining 32-39% of events being level 2 cases of hypoglycemia (<54 mg/dL) (FIG. 1b, e). The incidence rate of level 2 parent events in the general T1D population is on average 219 events per patient-year which is 1.5 to 2.5-fold higher than the 91 level 2 parent events per patient-year observed in people with well-controlled T1D. These results suggest that the incidence of hypoglycemia is common both during the day and at night, especially for level 1 cases. These rates are relatively low (i.e., 0.25-0.60 versus ˜1 events per patient per day), likely due to the treatment of initial and subsequent follow-on events as discrete events and differences in the hypoglycemic thresholds used in the analysis.

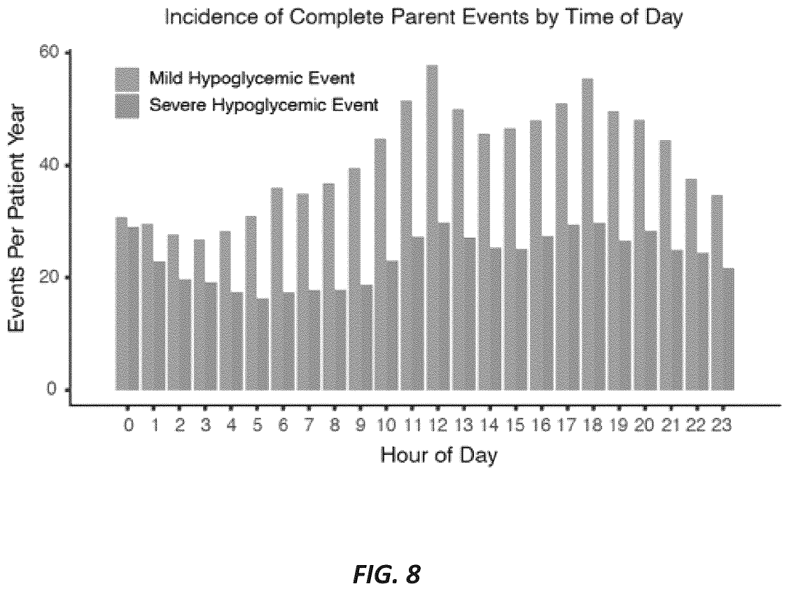

Classification of parent hypoglycemic events by time of occurrence reveal that 76-80% of events occur during the day, with the remaining 20-24% of events occurring at night (FIG. 6e). A spike in both level 1 and level 2 hypoglycemic parent events was observed at 12:00 (noon) and 18:00 hours, presumably due to meals, as well as at midnight (00:00) (FIG. 8).

### Hypoglycemia Onset: Rate and Time in Pre-Hypoglycemia

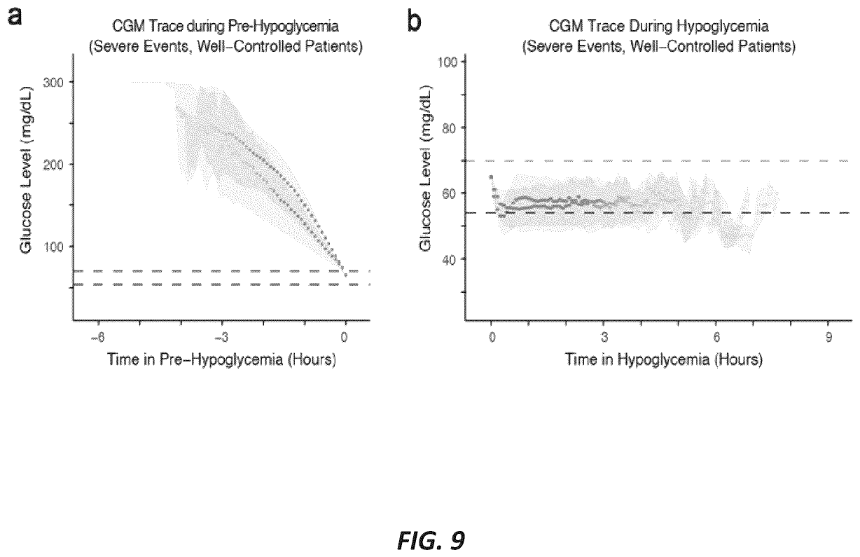

The pre-hypoglycemic period represents a window of opportunity to prevent impending hypoglycemia following the administration of excessive insulin and was found to differ based on the severity and time of occurrence (FIG. 5C and FIG. 9). On average, this duration was 14.3% to 17.3% shorter for level 2 events compared to level 1 events and was accompanied by faster rates of blood glucose decline (−2.54 to −5.2 mg/dL/min) compared to level 1 events (−0.88 to −1.05 mg/dL/min).

The pre-hypoglycemic duration for level 1 nocturnal hypoglycemic events was 6.5% longer than level 1 daytime events (p=1.369e⁻⁵). This may be due to the use of short-acting insulin during the day compared to the use of long-acting insulin before sleep. Notably, no substantial difference in the pre-hypoglycemic duration was observed between daytime and nighttime level 2 events (p=0.7667). Finally, the average duration of pre-hypoglycemia was found to be 29.2% to 75.8% longer in the general T1D population compared to patients with well-controlled disease (p<2.2e⁻¹⁶)

### Hypoglycemic Event Duration

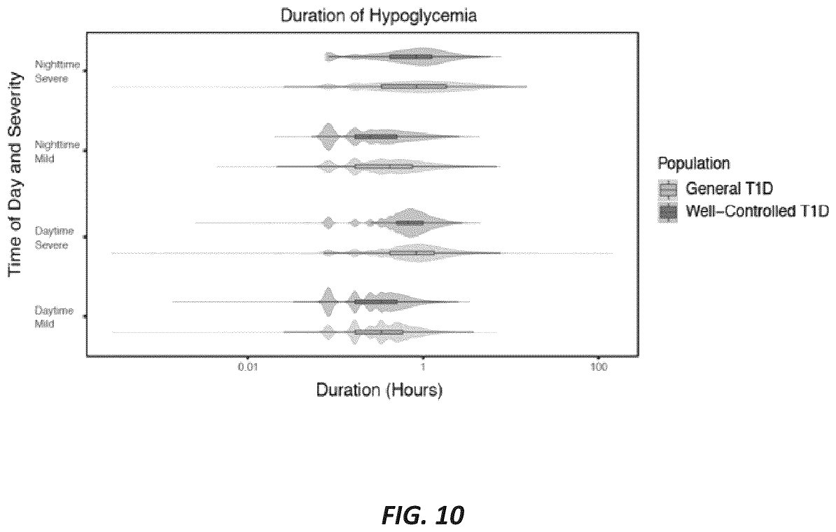

The various timings of hypoglycemic events represent multiple windows of opportunity for intervention including the opportunity to mitigate the progression of level 1 to level 2 hypoglycemia, and the opportunity to provide automated glucagon treatment of level 2 hypoglycemia. The aggregate CGM traces during hypoglycemia are shown in FIG. 9. The average duration of parent hypoglycemic events is dependent on severity and time of occurrence (FIG. 5C). During the day, the average level 1 hypoglycemic event lasted 35.27 minutes compared to the average level 2 hypoglycemic event which lasted 74.17 minutes, representing a 110.3% longer duration for level 2 events (p<2.2e⁻¹⁶) (FIGS. 6f and 10). During the night, the average level 1 hypoglycemic event lasted 49.77 minutes and the average level 2 hypoglycemic event lasted 108.38 minutes, representing a 117.8% longer duration for level 2 events (p<2.2e⁻¹⁶). On average, nocturnal hypoglycemic events were longer than daytime events (i.e., 41.1% for level 1 events, p=3.175e⁻¹⁶; and 46.1% for level 2 events, p<2.2e⁻¹⁶). Finally, the average duration of each hypoglycemic event (daytime level 1, daytime level 2, nighttime level 1, nighttime level 2) was between 35.3% and 108.4% longer in the general T1D population compared to patients with well-controlled disease (p<2.2e⁻¹⁶ for daytime level 1, daytime level 2, nighttime level 1; p=2.871e⁻¹² for nighttime level 2).

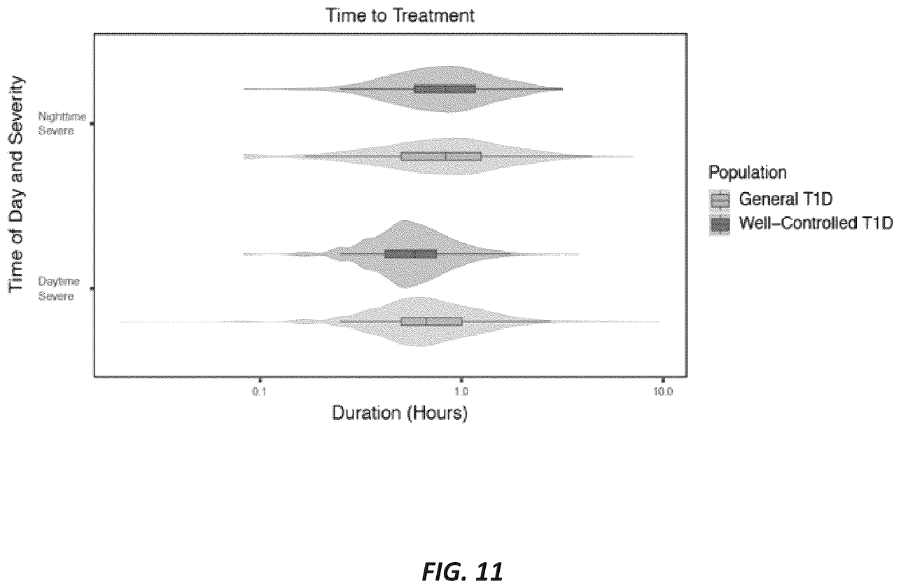

The average time to glucagon treatment of level 2 hypoglycemia in the general T1D population was 49.57 minutes during the day and 57.58 minutes during the night (p=7.2e⁻¹⁵) (FIG. 6g, and FIG. 11). A trend towards shorter treatment times was observed in patients with well-controlled T1D during the day (37.48 minutes, p=1.46e⁻¹²), but this difference was not significant at night (38.78 minutes, p=0.8339).

The duration of a hypoglycemic event consists of the time it takes for patients to recognize the onset of hypoglycemia, administer an intervention, and the time needed for that intervention to restore euglycemia. While any treatment aim to rescue a patient from hypoglycemia in as short a timeframe as possible, the reported durations reflect current medical practice and therefore a lower threshold for which a glucagon treatments should strive to restore euglycemia to be considered a viable treatment option.

### CGM Analysis

The analysis (1) characterizes hypoglycemia as distinct events rather than simplified ‘time-spent-in hypoglycemia’ or ‘percent nights with hypoglycemia’ parameterizations, (2) provides holistic treatment of the data by including pre-hypoglycemic periods and associated follow-on hypoglycemic events in the analysis, (3) extracts pertinent features for identifying windows of opportunity for glucagon intervention including time-to-treatment, duration and rate of hypoglycemia onset, duration in level 1 hypoglycemia before level 2 hypoglycemia, and duration of hypoglycemic events, and (4) expands this analysis to include different levels of hypoglycemia severity, different times of occurrence, and both well-controlled and general T1D patient populations.

Results from the analysis show that a relatively large proportion (i.e., 20-30%) of hypoglycemic episodes are follow-on events, suggesting that only 70-80% of all complete events are independent and the total duration of these independent events may be longer when accounting for the associated follow-on events. Roughly 15-20% of parent events were associated with one follow-on event, and parent events with more than one follow-on event occur at a progressively lower frequency. This may suggest that under-treatment of an initial hypoglycemic event leads to failure to maintain euglycemia. Patients with well-controlled diabetes experience ˜14% fewer independent hypoglycemic events compared to the general T1D population, and these instances are associated with ˜10% fewer follow-on events suggesting that patients with well-controlled T1D experience higher success at maintaining euglycemia following an initial hypoglycemic event.

Analysis of hypoglycemic event trajectories in the CGM data shows that the rate and duration of hypoglycemia onset, duration of hypoglycemia, and time to hypoglycemia treatment differ based on event severity (level 1 vs. level 2 hypoglycemia), time of hypoglycemia onset (daytime vs. nighttime), and patient population (general vs. well-controlled disease). The rate of glucose decline leading to hypoglycemia onset was found to be faster for daytime events compared to nocturnal events, and in level 2 cases compared to level 1 cases. The former may be due to exercise or the use of rapid-acting insulins during the day while the latter may be a direct result of the dose-dependent effect of insulin which affects both the degree and rate of glucose decline. Surprisingly, these differences only translated to small changes in the average pre-hypoglycemia duration, which ranged between 1.0 hours to 1.3 hours in the general T1D population and 44 to 47 minutes in the well-controlled T1D population. This pre-hypoglycemic period represents a largely invariable 44 or 60-minute window of opportunity to prevent impending hypoglycemia following the administration of excessive insulin, either through glucagon mini-dose treatment or other interventions.

In comparison, the average duration of level 1 hypoglycemia preceding the onset of a level 2 hypoglycemic event was found to be significantly longer during the night (i.e., ˜16 minutes versus 25 minutes), and represents the second window of opportunity for level 2 hypoglycemia prevention once level 1 hypoglycemia has been detected. This period is relatively notable for daytime events where glucagon mini doses may be manually administered shortly after the patient is alerted to low glucose levels to mitigate the risk of a level 2 event.

The base utility of glucose-responsive or closed-loop glucagon interventions is to provide automated glucagon treatment faster than what is achieved using manual intervention. This window of opportunity is highly variable. In general, the average duration of parent hypoglycemic events is longer for level 2 cases compared to level 1 cases, during nocturnal events compared to daytime events, and in the general T1D population compared to subjects with well-controlled T1D. These durations can be substantial, ranging from 26 minutes for daytime level 1 hypoglycemia in subjects with well-controlled disease to 1 hour and 48 minutes for level 2 nocturnal hypoglycemia in the general T1D patient population.

The average time to glucagon administration, if detected, ranged from −37.5 minutes in patients with well-controlled diabetes to over 50 minutes in the general population (although this difference was not significant for nighttime events). In some instances, multiple glucagon administration events were observed, suggesting that these patients had trouble maintaining euglycemia following a single bolus administration of glucagon. The average time to last glucagon treatment ranged between 53.5 minutes to over 1 hour. This suggests at least in-part that patients can struggle for prolonged periods of time to control glycemic levels. These observations highlight the clinical utility of automated glucagon system with the ability to provide either multiple cycles or persistent glucagon delivery following hypoglycemia-triggering.

### Methods for CGM Analysis

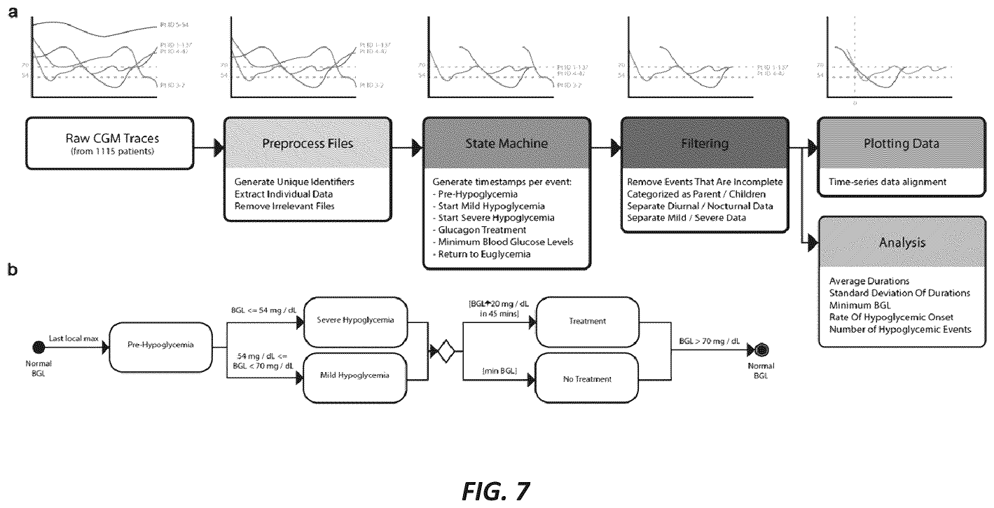

The method for CGM analysis is generally described in FIG. 7. A retrospective analysis of continuous glucose monitoring (CGM) data from five publicly available clinical trial datasets was performed to characterize the kinetics of hypoglycemia onset, progression, and resolution in patients with type 1 diabetes. The aggregated dataset is comprised of over 89855 days (approximately 79 days per patient) of CGM traces from 1115 patients that are equally represented in sex (50.9% female; 49.1% male) and highly inclusive across all age groups ranging from 3 to 79 years of age. The dataset was divided by patient enrollment criteria adopted by each of the 5 studies. Data from 909 patients are aggregated from 4 datasets and are considered to be representative of the general type 1 diabetic population. The remaining 226 patients enrolled in the study are considered representative of type 1 diabetes patients with well-controlled diabetes having no recent history of hypoglycemia or hypoglycemia unawareness.

### Extracting Hypoglycemic Events

The processing pipeline had five stages: (1) preprocessing the raw CGM traces, (2) running the traces through a state machine to extract hypoglycemic events, (3) filtering the events to create a clean dataset, and lastly, (4) analysis and (5) plotting the events. Stages 1-4 were conducted using Python 3.9. All statistical tests and plotting of data were completed using RStudio.

To preprocess the raw data, unique identifiers were generated across datasets from the raw CGM to prevent patient data overlap between studies. Individual data was extracted into separate files, removing files that do not have BGL data below 70 mg/dL to streamline the analysis and reduce memory consumption.

The preprocessed data was run through a state machine to capture complete hypoglycemic events. Each complete event is represented by a CGM trace comprised of (1) a pre-hypoglycemic period, defined as the period starting at the first local maximum preceding a steady decline in glucose levels into hypoglycemia and concluding when glucose levels drop below the hypoglycemic threshold of 70 mg/dL, and (2) the hypoglycemic period during which blood glucose levels remain below 70 mg/dL. The hypoglycemic period may be further characterized by a period of level 1 hypoglycemia wherein glucose levels remain between 54 mg/dL and 70 mg/dL, and in some cases, a period of level 2 hypoglycemia wherein glucose levels drop below 54 mg/dL. A hypoglycemic recovery period is defined by a steady rise in glucose levels from a local minimum back above the 70 mg/dL hypoglycemic threshold to euglycemia. Recovery may be due to the administration of exogenous glucagon, as identified in the CGM trace by a rapid and persistent increase in glucose levels exceeding 20 mg/dL within a 45-minute interval, or for other reasons (e.g., ingestion of carbohydrates, etc.). Analysis parameters extracted from each hypoglycemic event include the time of onset and resolution for pre-hypoglycemia, level 1 hypoglycemia, and level 2 hypoglycemia; the initial glucose level at the start of the pre-hypoglycemic period; the minimum glucose level during the event; and the time of glucagon treatment if detected.

CGM traces in which glucose levels do not return to euglycemia (70 mg/dL) due to truncation or gaps in the data are deemed incomplete and were excluded from the analysis (22976 cases; 14270 well-controlled cases; 8706 general population cases). 135,386 distinct complete hypoglycemic events were identified in total. The general population generated 95,953 distinct complete hypoglycemic events, while the well-controlled population generated 62,391.

These remaining complete events were categorized into ‘parent’ events, which can be comprised of individual standalone events or as a series of multiple events. Standalone complete hypoglycemic events or the first event in a series were defined as ‘initial’ events, while hypoglycemic events that closely follow a preceding event within a time interval of <=1 hour were categorized as ‘follow-on’ events. Each ‘parent’ event was categorized by the time (i.e., daytime vs. nighttime events) and the severity (i.e., level 1 vs. level 2 hypoglycemia events) of the minimum glucose level of the ‘initial’ hypoglycemia occurrence. Events in which the onset of the ‘initial’ hypoglycemia occurs between 11 μm and 5:59:59 am are considered night-time events while those with onset times between 6 am and 10:59:59 μm are considered daytime events.

### Hypoglycemic Event Analysis

Clinical parameters were calculated for each event including the incidence rate, the duration of hypoglycemia, time to glucagon treatment, and the rate of hypoglycemia onset. Average duration refers to the average duration in hypoglycemia across a parent event (including parent and follow-on events). Average pre-hypo duration includes the “pre-hypoglycemic” phase for only the initial event in a parent event. Average duration to the first glucagon treatment refers to the time between when a patient first drops into hypoglycemia to the time of first treatment in a parent event. Average duration to last glucagon treatment refers to the time between when a patient first drops into hypoglycemia to the time of last treatment in a parent event. Average level 1 duration before first level 2 hypoglycemia refers to the time between when a patient drops into hypoglycemia to when they first drop into level 2 hypoglycemia. Average pre-hypoglycemia to first level 2 hypoglycemia refers to the time between a patient's pre-hypo period to the first time they drop into level 2 hypoglycemia. The rate of hypoglycemia onset refers to the difference in glucose levels between the start time of hypoglycemia and pre-hypoglycemia divided by the time taken to drop into hypoglycemia. Average min BGL refers to the minimum blood glucose level across the entire event.

### Statistical Analysis of Hypoglycemic Events

The statistical significance of the clinical parameters generated in the analysis between well-controlled and general populations was investigated. Additionally, the statistical significance of the clinical parameters between the individual datasets to capture inter-dataset differences was calculated. Clinical parameters did not appear to display normal distributions when plotted. Kruskal-Wallis was used as a non-parametric alternative to one-way ANOVA to compare population mean ranks.

### Confirming Data Analysis Stability

To evaluate the stability of the results, 10 Monte Carlo simulations were conducted for each of the 5 datasets, introducing noise mimicking the soft failure of the individual devices. Assuming the data is stable, the trends and rates from the simulated data should be similar to the output of the original analysis.

Each dataset contains numerous individual traces from a device (min: 6351, max: 1715152). The noise for trace i within a dataset was sampled using Equation 1.

Noise_i˜N(μ,σ) Equation 1.

where μ is zero and σ is the MARD score of the study-specific device as documented in the literature⁵¹⁻⁵⁴. The simulated signal at time t for trace i within a simulation run was then calculated using Equation 2.

$${Simulatedsignal}_{i} , t = {Gl}_{i , t}^{Actual} + {Noise}_{i} , \qquad Equation 2.$$

Where Gl_{i,t}^{Actual} is the original glucose datapoint for trace i at time t. After regenerating a complete dataset of simulated signals, the procedures outlined above to recalculate the durations, rates, and trends of hypoglycemia.

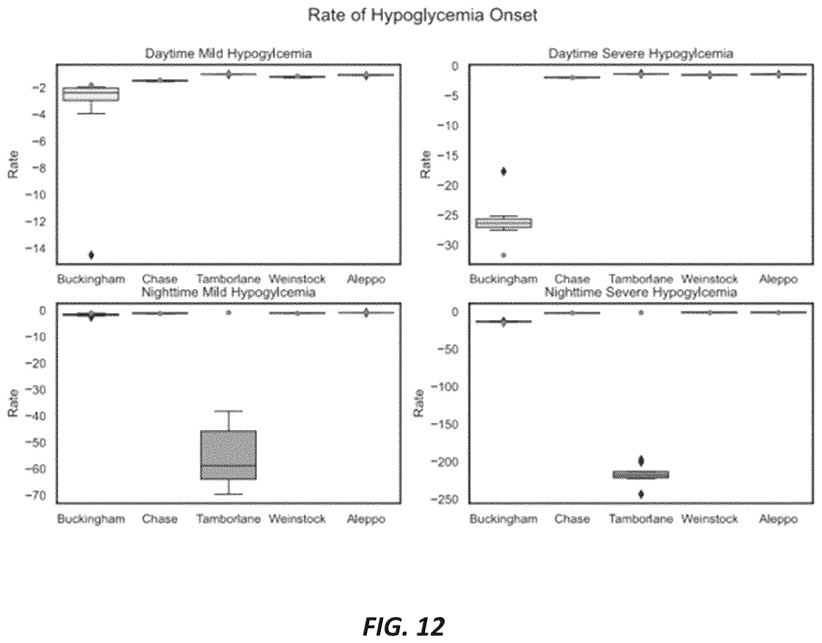

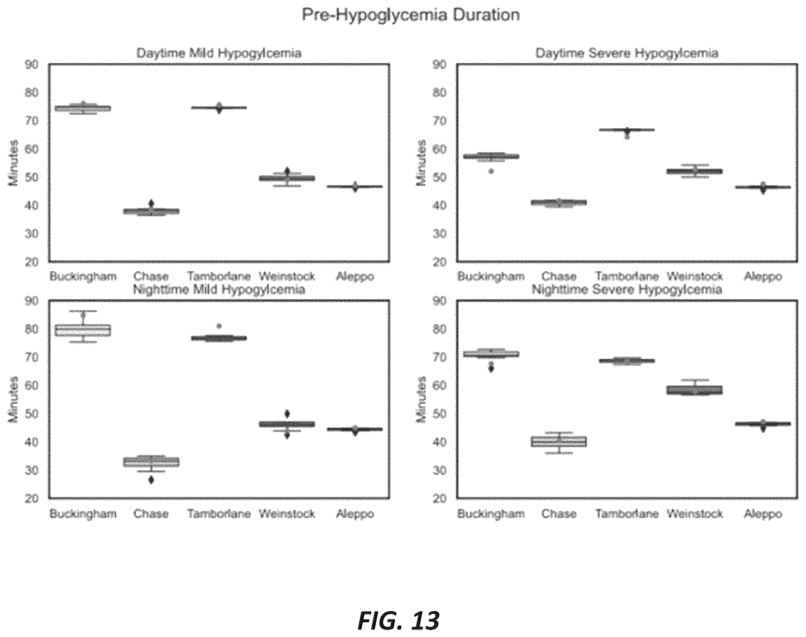

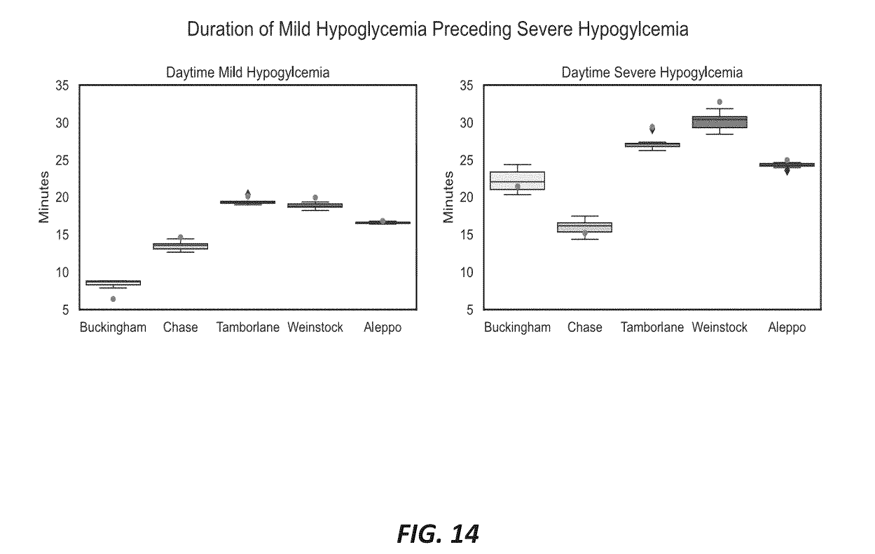

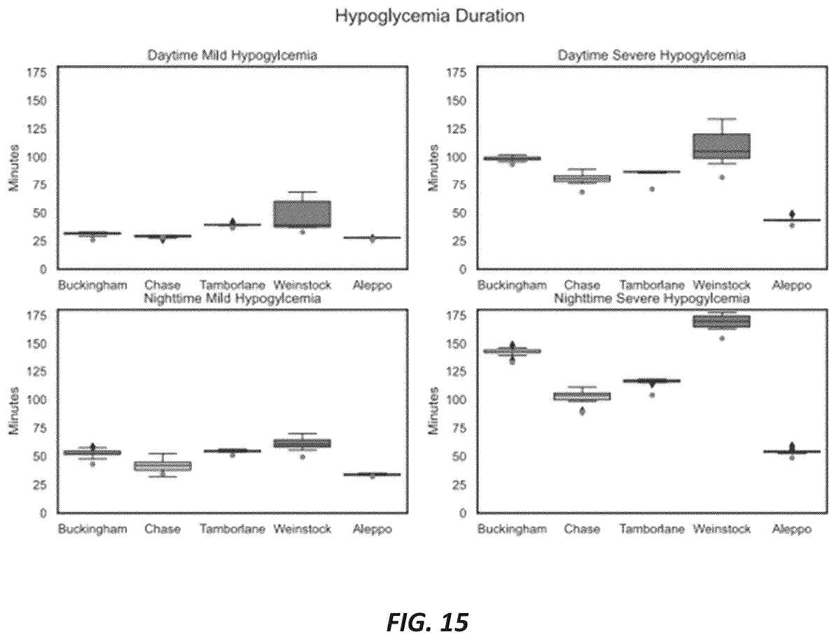

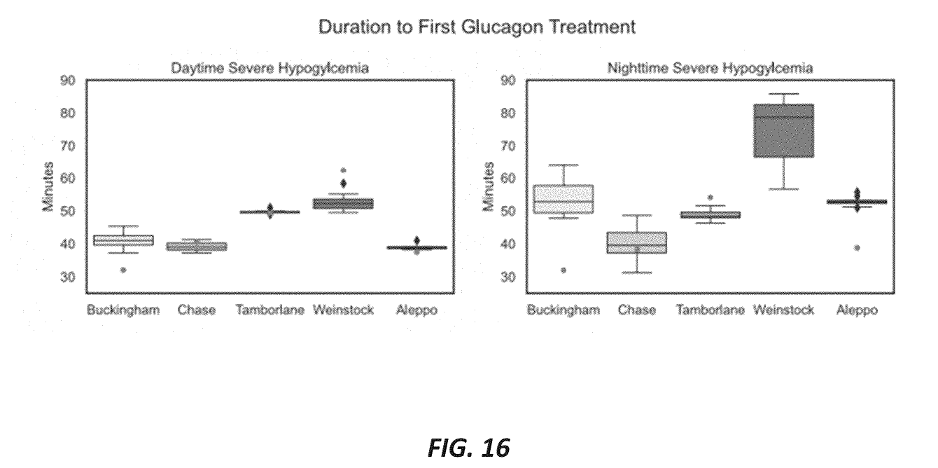

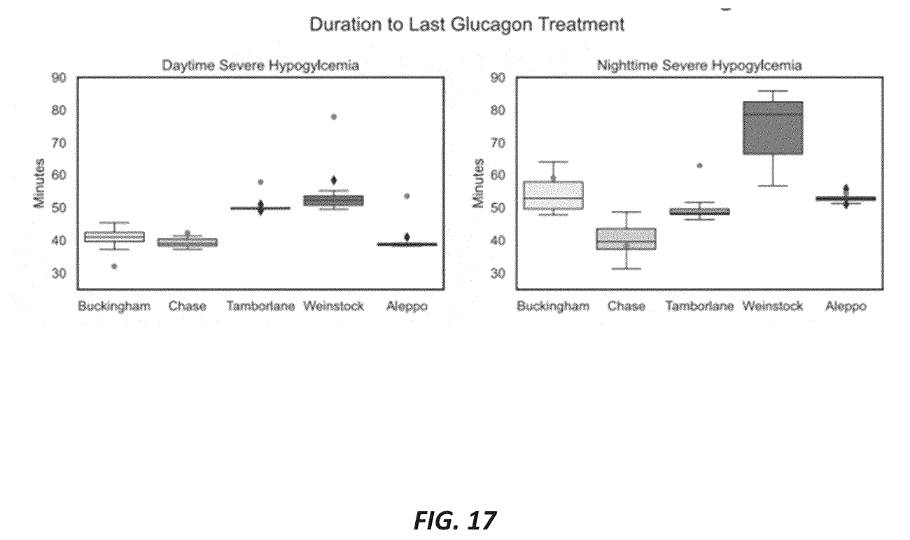

The plots suggest that the raw data is relatively stable, and, importantly, the simulations support the differences between subjects with well-controlled T1D and general T1D. However, there are instances where the simulation results deviate from the analyzed raw data. For example, the simulated daytime level 2 hypoglycemia durations in the Tamborlane study trend 20 minutes longer than in the raw data. In this case, it further supports that there is a distinction between the general and well-controlled populations, perhaps one that is worse than reported above. Additionally, the first glucagon treatment—nighttime level 2 (FIGS. 16-17) and the last glucagon treatment—daytime level 2 (FIGS. 16-17) durations, the Aleppo study's simulated and raw data deviate by approximately 10 minutes. In FIG. 16, the durations between daytime and nighttime glucagon treatments for populations with well-controlled diabetes (Aleppo) demonstrate an increase in distinction than reported above. The above results are further described in FIGS. 12-17.

### Related Materials and Methods

The following paragraphs describes related materials and methods used to generate and/or evaluate experimental data presented elsewhere in this disclosure.

Dulbecco's Phosphate-Buffered Saline (PBS) was purchased from Gibco by Life Technologies (Woburn, USA). Recombinant human insulin, glucagon, and Insulin Degludec was obtained from Novo Nordisk A/S (Maalov, Denmark). Steel microneedle masters were purchased from Kjul & Co, ApS (Brondby, Denmark). Shellac (Protect EN RX Cat #509521020, Lots 5688996 and 5550391) was gifted and purchased from Sensient Pharmaceutical Technologies (St Louis, MO, USA). Pharmaceutical grade 280 kDa hyaluronic acid was gifted from Bloomage Freda Biopharm Co. (Jinan, China). Dextrose, porcine gelatin (Type A), sorbitol, and streptozotocin were purchased from Sigma Aldrich (Saint Louis, USA).

### Synthesis of Gelatin Methacrylate.

20 g of gelatin (porcine, type A) was dissolved in 20 mL of 60° C. DI water. 10 mL of methacrylic anhydride was added dropwise to the stirred solution. The reaction was allowed to proceed for 2 hours at 60° C. The resulting product was cooled to room temperature and dialyzed against DI water for 3 days at 4° C.

### Synthesis of Methacrylated Hyaluronic Acid.

2 g of HA was dissolved in 100 mL of DI water and stirred at 500 rpm for 2 hours at room temperature. 1.6 mL of methacrylic anhydride was added dropwise to the solution. 5 N NaOH was added dropwise to adjust the final pH of the solution to pH 8.5. The solution was protected from light and stirred at 4° C. for 24 hours. 2.92 g of NaCl was added to the solution followed by precipitation of the polymer in ethanol. The resulting product was then isolated by centrifugation and washed in ethanol. The product was then redissolved in DI water and dialyzed against DI water for 3 days at 4° C.

### In Vivo Evaluation of Glucagon Formulations in Diabetic Rats

200-225 g Sprague Dawley (SAS SD strain 400) instrumented with jugular vein catheters and Instech buttons were purchased from Charles River. The animals were starved for 8 hours and subsequently treated with streptozotocin (STZ) solution, freshly prepared in pH 4.5 50 mM citrate buffer, at a 65 mg/kg dose and administered by intraperitoneal injection.

A 1 U/mL stock solution of human recombinant insulin was prepared by dissolving 1.36 mg of human recombinant insulin in 23.0 mL of pH 8.0 sterile saline. A 1 U/mL stock solution of insulin Degludec was prepared by diluting 100 μl of commercial U100 (100 U/mL) Degludec in 9900 μl of sterile saline. A 1 U/mL stock of glucagon solution was prepared by diluting 1 mg of glucagon in 1 mL of sterile saline supplemented with HCl to a final pH of 3.0). Animals were fasted for 8 hours before treatment with a total insulin dose of 2-6 U/kg, made up of 30% human recombinant insulin and 70% insulin Degludec, to induce stable euglycemia or hypoglycemia. Insulin formulations were administered in separate subcutaneous injections away from the site of patch application. Stable euglycemia or hypoglycemia was achieved 3-hours following insulin dosing. Patches were applied to the shaved and de-pilfered skin of the animals and secured using a Tegaderm bandage. Acute patches were removed 5 minutes after application while responsive patches were left in place for the duration of the experiment. Control animals were treated with freshly prepared glucagon solution at a dose of 300 μg of glucagon via SC injection. At various timepoints, blood was sampled from the jugular catheter, measured for blood glucose levels using a handheld glucometer, and collected in BD microtainer tubes with K₂EDTA and 14 μL of 7 TIU/mL (91 kIU/mL, lot SLBT2024) aprotinin. Samples were stored on ice prior to processing. Each sampled was centrifuged at 10,000 rpm for 5 minutes and the plasma was collected and stored at −80° C. prior to analysis. Plasma glucagon and c-peptide levels were measured using commercial ELISA kits (Invitrogen) according to the manufacturer's protocols.

### Statistical Analysis

Unpaired two-sided Student's t-tests and one-way analysis of variance (ANOVA) with Tukey's multiple comparisons tests were performed using GraphPad Prism (Version 9.4.1) or Microsoft Excel.

### CONCLUSION

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain, using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A glucose-responsive system for delivering glucagon to a mammalian subject, the glucose-responsive system comprising:
a polymeric release structure comprising:
a readily soluble and thermostable (ReST) glucagon formulation;
at least one of glucose oxidase, glucose oxidase derivative, or glucose oxidase analogue; and
a pH-responsive polymer,
wherein the polymeric release structure swells and/or dissolves at a pH level of about 6 or greater, and
wherein glucagon is released from the polymeric release structure when the mammalian subject has a glucose concentration of about 100 milligrams per deciliter or less.

2. The glucose-responsive system of claim 1, wherein the ReST glucagon formulation consists essentially of an excipient and at least one of glucagon, a glucagon analog, or a glucagon derivative.

3. The glucose-responsive system of claim 2, wherein the ReST glucagon formulation comprises about 0.2 mg to about 2.0 mg of the at least one of the glucagon, the glucagon analog, or the glucagon derivative.

4. The glucose-responsive system of claim 2, wherein the ReST glucagon formulation comprises about 10% to about 90% of the at least one of the glucagon, the glucagon analog, or the glucagon derivative.

5. The glucose-responsive system of claim 2, wherein the excipient comprises one or more of benzoic acid, sodium carbonate, meglumine, sodium phosphate tribasic, myristyl sulfobetaine, Kollidon HS 15, dodecylphosphocholine, L-Glutamine, PIPES, and/or sodium succinate.

6. The glucose-responsive system of claim 1, wherein the at least one of glucose oxidase, glucose oxidase derivative, or glucose oxidase analogue is incorporated into the pH-responsive polymer.

7. The glucose-responsive system of claim 1, wherein the polymeric release structure further comprises at least one of catalase, catalase derivative, catalase analogue, or MnO₂.

8. The glucose-responsive system of claim 1, wherein the pH-responsive polymer comprises one of shellac, methyl acrylate-methacrylic acid copolymers, cellulose acetate phthalate, cellulose acetate succinate, hydroxypropyl methyl cellulose phthalate, hydroxypropyl methyl cellulose acetate succinate, polyvinyl acetate phthalate, methyl methacrylate-methacrylic acid copolymers, cellulose acetate trimellitate, or zein.

9. The glucose-responsive system of claim 1, further comprising:
a hydrogel layer loaded with the at least one of glucose oxidase, glucose oxidase derivative, or glucose oxidase analogue and disposed on the pH-responsive polymer.

10. The glucose-responsive system of claim 1, wherein the pH-responsive polymer forms an enteric barrier film configured to swell and/or dissolve at the pH level of about 6 or greater.

11. The glucose-responsive system of claim 10, wherein the enteric barrier film has a thickness of about 10 microns to about 400 microns.

12. The glucose-responsive system of claim 10, wherein the at least one of glucose oxidase, glucose oxidase derivative, or glucose oxidase analogue is configured to catalyze production of hydrogen peroxide and gluconic acid in the presence of glucose and oxygen, thereby keeping the pH level below 6 and preventing the enteric barrier film from swelling or dissolving.

13. The glucose-responsive system of claim 12, wherein the polymeric release structure further comprises at least one of catalase, catalase derivative, catalase analogue, or MnO₂ to catalyze production of oxygen from the hydrogen peroxide.

14. The glucose-responsive system of claim 1, wherein the polymeric release structure is formed into a microneedle of a microneedle transdermal patch, wherein the glucagon is released from at least one of an interior bore of the microneedle or an outer surface of the microneedle.

15. The glucose-responsive system of claim 1, wherein the polymeric release structure is formed into an implantable structure configured to be implanted into the mammalian subject beneath a stratum cornea of the mammalian subject using a hypodermic needle or trocar.

16. The glucose-responsive system of claim 1, wherein the polymeric release structure is formed into a microparticle configured to be implanted into the mammalian subject beneath a stratum cornea of the mammalian subject using a hypodermic needle or trocar.

17. A method of delivering glucagon to a mammalian subject with a release structure comprising a readily soluble and thermostable (ReST) glucagon formulation contained within a pH-sensitive polymer that incorporates or is coated with at least one of glucose oxidase, glucose oxidase derivative, or glucose oxidase analogue and that swells and/or dissolves at a pH of about 6 or greater, the method comprising:
inserting the release structure in the mammalian subject or applying the release structure to skin of the mammalian subject,
wherein the at least one of glucose oxidase, glucose oxidase derivative, or glucose oxidase analogue catalyzes production of gluconic acid and hydrogen peroxide in the presence of glucose and oxygen, thereby keeping the pH of the pH-sensitive polymer below 6, in the presence of glucose and preventing release of the ReST glucagon formulation from the release structure, and
wherein the pH of the pH-sensitive polymer rises above 6 in the absence of glucose, causing the pH-sensitive polymer to swell and/or dissolve, thereby releasing the ReST glucagon formulation from the release structure.

18. The method of claim 17, wherein releasing the ReST glucagon formulation from the release structure occurs at a rate of about 1 mg in 30 seconds to about 1 mg in 4 hours.

19. The method of claim 17, wherein the release structure further comprises at least one of catalase, catalase derivative, catalase analogue, or MnO₂ to catalyze production of oxygen from the hydrogen peroxide.

20. A readily soluble and thermostable glucagon formulation consisting essentially of:
at least one of glucagon, a glucagon analog, or a glucagon derivative; and
an excipient from the group consisting of benzoic acid, sodium carbonate, meglumine, sodium phosphate tribasic, myristyl sulfobetaine, Kollidon HS 15, dodecylphosphocholine, L-Glutamine, PIPES, or sodium succinate.
